(19) United States
(12) Patent Application Publication
(10) Pub. No.: US 2017/0248587 A1
(43) Pub. Date: Aug. 31, 2017

(54) Polymeric Dye Specific Binding Members and Methods of Making and Using the Same

(71) Applicant: Becton, Dickinson and Company, Franklin Lakes, NJ (US)

(72) Inventors: James Ghadiali, San Diego, CA (US); Jody Martin, Encinitas, CA (US)

(73) Assignee: Becton, Dickinson and Company, Franklin Lakes, NJ (US)

(21) Appl. No.: 15/509,827

(22) PCT Filed: Nov. 11, 2015
(86) PCT No.: PCT/US2015/060206

§ 371 Date: Mar. 8, 2017

Related U.S. Application Data
(60) Provisional application No. 62/078,890, filed on Nov. 12, 2014.

(51) Int. Cl. G01N 33/533; G01N 33/569; G01N 33/543; G01N 33/58

(52) U.S. Cl. CPC G01N 33/533; G01N 33/582; G01N 33/56972; G01N 33/54326

(57) ABSTRACT

Proteinaceous specific binding members that specifically bind to a polymeric dye are provided. Also provided are methods of using the specific binding members, e.g., in separating a polymeric dye-labeled cell from a sample, in analyte detection, etc., as described herein. Kits and systems for practicing the subject methods are also provided.

### CROSS-REFERENCE TO RELATED APPLICATIONS

This application is related to U.S. Provisional Patent Application Ser. No. 62/078,890, filed Nov. 12, 2014, the disclosure of which application is incorporated herein by reference.

### INTRODUCTION

Molecular recognition involves the specific binding of two molecules. The ability to manipulate the interactions of such molecules is of interest for both basic biological research and for the development of therapeutics and diagnostics. Pairs of molecules which have binding specificity for one another find use in a variety of research and diagnostic applications, such as the labeling and separation of analytes, flow cytometry, in situ hybridization, enzyme-linked immunosorbent assays (ELISAs), western blot analysis, magnetic cell separations and chromatography. Members of specific binding pairs can be found in a variety of different types of molecules. For example, antibodies are a class of protein that has yielded specific binding ligands for various target antigens, such as proteins, peptides and small molecules. For example, nonimmunological binding pairs include biotin-streptavidin, hormone-hormone binding protein, receptor-receptor agonist or antagonist, IgG-protein A, lectin-carbohydrate, enzyme-enzyme cofactor, enzyme-enzyme-inhibitor, and complementary polynucleotide pairs capable of forming nucleic acid duplexes.

### SUMMARY

Proteinaceous specific binding members that specifically bind to a polymeric dye are provided. Also provided are methods of using the specific binding members, e.g., in separating a polymeric dye-labeled cell from a sample, in analyte detection, etc., as described herein. Kits and systems for practicing the subject methods are also provided.

### BRIEF DESCRIPTION OF THE FIGURES

It is understood that the drawings, described below, are for illustration purposes only. The drawings are not intended to limit the scope of the present teachings in any way.

FIG. 1 shows antibody isotype test results for clones selected for reactivity against a polymeric dye.

FIG. 2 illustrates schematics of components of interest that find use in an embodiment of a subject method of separating a cell. Target cell (100) has a lineage-specific marker (101) on the cell surface. The polymeric dye labeled affinity agent (200) is composed of an affinity agent (e.g., an antibody, 201) that specifically binds the lineage-specific cell marker (101) conjugated to a polymeric dye (202). Support-bound proteinaceous specific binding member (300) is composed of a proteinaceous specific binding member (301) that specifically binds the polymeric dye (201) and a solid support (302).

FIG. 3 illustrates steps of interest in an embodiment of a subject method of separating a cell: (A) labeling of the target cell (100) with a polymeric dye labeled affinity agent (200) (e.g., a lineage specific antibody conjugated to a polymeric dye) and capturing of the target cell with a support-bound proteinaceous specific binding member (300) (e.g., a magnetic particle bound anti-polymeric dye antibody); (B) application of the external magnetic field of a magnet (400) to retain magnetic particle bound cells (100), where non-binding cells (102) are washed away; and (C) release of cells (100) from the magnetic particles using a biocompatible elution buffer to produce purified and isolated cells.

FIGS. 4-7 illustrate the selection and capture of specific cell subtypes and subsequent release of particle-bound cells. Peripheral blood mononuclear cells were stained with an anti CD3-BV421 (Brilliant Violet 421™) conjugate followed by red blood cell lysis. The sample was contacted with anti-BV421 bound to magnetic particles. Magnetically-labeled components were separated using a magnet and the bound and unbound cell fractions analyzed by flow cytometry. The magnetic particle-bound cells were subsequently treated with a biocompatible elution buffer and exposed to the external magnetic field of a magnet to remove the liberated magnetic particles and yield a purified particle-free cell population. FIG. 4 shows analysis of a sample including anti-CD3-BV421 labelled peripheral blood mononuclear cells. FIG. 5 shows analysis of CD3 positive cells magnetically depleted from the sample using anti BV421 coated magnetic particles (negative selection). FIG. 6 shows an analysis of magnetically enriched CD3 positive cells, bound to magnetic particles (positive selection), where the light scattering profile indicates particles remain bound to the cell surface. FIG. 7 shows an analysis of magnetically enriched CD3 positive cells, subsequently released from magnetic particles, as indicated by the light scattering profile.

FIGS. 8 and 9 illustrate the capture of CD3 positive lymphocytes from whole blood without additional lysis. FIG. 8 shows the light scattering and fluorescence emission intensity of magnetic particle-bound lymphocytes. FIG. 9 shows the light scattering and fluorescence emission intensity of CD3.

FIG. 10 provides an illustration of an energy transfer assay as described in greater detail in the Experimental section, below.

### DEFINITIONS

Before describing exemplary embodiments in greater detail, the following definitions are set forth to illustrate and define the meaning and scope of the terms used in the description.

Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Markham, THE HARPER COLLINS DICTIONARY OF BIOLOGY, Harper Perennial, N.Y. (1991) provide one of skill with the general meaning of many of the terms used herein. Still, certain terms are defined below for the sake of clarity and ease of reference.

It must be noted that as used herein and in the appended claims, the singular forms “a”, “an”, and “the” include plural referents unless the context clearly dictates otherwise. For example, the term “a primer” refers to one or more primers, i.e., a single primer and multiple primers. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

As used herein, the term “sample” relates to a material or mixture of materials, in some cases in liquid form, containing one or more analytes of interest. In some embodiments, the term as used in its broadest sense, refers to any plant, animal or bacterial material containing cells or producing cellular metabolites, such as, for example, tissue or fluid isolated from an individual (including without limitation plasma, serum, cerebrospinal fluid, lymph, tears, saliva and tissue sections) or from in vitro cell culture constituents, as well as samples from the environment. The term “sample” may also refer to a “biological sample”. As used herein, the term “a biological sample” refers to a whole organism or a subset of its tissues, cells or component parts (e.g. body fluids, including, but not limited to, blood, mucus, lymphatic fluid, synovial fluid, cerebrospinal fluid, saliva, amniotic fluid, amniotic cord blood, urine, vaginal fluid and semen). A “biological sample” can also refer to a homogenate, lysate or extract prepared from a whole organism or a subset of its tissues, cells or component parts, or a fraction or portion thereof, including but not limited to, plasma, serum, spinal fluid, lymph fluid, the external sections of the skin, respiratory, intestinal, and genitourinary tracts, tears, saliva, milk, blood cells, tumors and organs. In certain embodiments, the sample has been removed from an animal or plant. Biological samples may include cells. The term “cells” is used in its conventional sense to refer to the basic structural unit of living organisms, both eukaryotic and prokaryotic, having at least a nucleus and a cell membrane. In certain embodiments, cells include prokaryotic cells, such as from bacteria. In other embodiments, cells include eukaryotic cells, such as cells obtained from biological samples from animals, plants or fungi.

As used herein, the terms “affinity” and “avidity” have the same meaning and may be used interchangeably herein. “Affinity” refers to the strength of binding, increased binding affinity being correlated with a lower Kd.

As used herein, the terms “determining,” “measuring,” and “assessing,” and “assaying” are used interchangeably and include both quantitative and qualitative determinations.

As used herein, the term “polypeptide” refers to a polymeric form of amino acids of any length, including peptides that range from 2-50 amino acids in length and polypeptides that are greater than 50 amino acids in length. The terms “polypeptide” and “protein” are used interchangeably herein. The term “polypeptide” includes polymers of coded and non-coded amino acids, chemically or biochemically modified or derivatized amino acids, and polypeptides having modified peptide backbones in which the conventional backbone has been replaced with non-naturally occurring or synthetic backbones. A polypeptide may be of any convenient length, e.g., 2 or more amino acids, such as 4 or more amino acids, 10 or more amino acids, 20 or more amino acids, 50 or more amino acids, 100 or more amino acids, 300 or more amino acids, such as up to 500 or 1000 or more amino acids. “Peptides” may be 2 or more amino acids, such as 4 or more amino acids, 10 or more amino acids, 20 or more amino acids, such as up to 50 amino acids. In some embodiments, peptides are between 5 and 30 amino acids in length.

As used herein the term “isolated,” refers to an moiety of interest that is at least 60% free, at least 75% free, at least 90% free, at least 95% free, at least 98% free, and even at least 99% free from other components with which the moiety is associated with prior to purification.

As used herein, the term “encoded by” refers to a nucleic acid sequence which codes for a polypeptide sequence, wherein the polypeptide sequence or a portion thereof contains an amino acid sequence of 3 or more amino acids, such as 5 or more, 8 or more, 10 or more, 15 or more or 20 or more amino acids from a polypeptide encoded by the nucleic acid sequence. Also encompassed by the term are polypeptide sequences that are immunologically identifiable with a polypeptide encoded by the sequence.

A “vector” is capable of transferring gene sequences to target cells. As used herein, the terms, “vector construct,” “expression vector,” and “gene transfer vector,” are used interchangeably to mean any nucleic acid construct capable of directing the expression of a gene of interest and which can transfer gene sequences to target cells, which can be accomplished by genomic integration of all or a portion of the vector, or transient or inheritable maintenance of the vector as an extrachromosomal element. Thus, the term includes cloning, and expression vehicles, as well as integrating vectors.

An “expression cassette” includes any nucleic add construct capable of directing the expression of a gene/coding sequence of interest, which is operably linked to a promoter of the expression cassette. Such cassettes can be constructed into a “vector,” “vector construct,” “expression vector,” or “gene transfer vector,” in order to transfer the expression cassette into target cells. Thus, the term includes cloning and expression vehicles, as well as viral vectors.

A “plurality” contains at least 2 members. In certain cases, a plurality may have 10 or more, such as 100 or more, 1000 or more, 10,000 or more, 100,000 or more, 10⁶ or more, 10⁷ or more, 10⁸ or more or 10⁹ or more members.

Numeric ranges are inclusive of the numbers defining the range.

The term “separating”, as used herein, refers to physical separation of two elements (e.g., by size or affinity, etc.) as well as degradation of one element, leaving the other intact.

As used herein, the term “specific binding” refers to the ability of a capture agent (or a first member of a specific binding pair) to preferentially bind to a particular analyte (or a second member of a specific binding pair) that is present, e.g., in a homogeneous mixture of different analytes. In some instances, a specific binding interaction will discriminate between desirable and undesirable analytes in a sample with a specificity of 10-fold or more for a desirable analyte over an undesirable analytes, such as 100-fold or more, or 1000-fold or more. In some cases, the affinity between a capture agent and analyte when they are specifically bound in a capture agent/analyte complex is at least 10⁻⁸M, at least 10⁻⁹M, such as up to 10⁻¹⁰M.

The methods described herein include multiple steps. Each step may be performed after a predetermined amount of time has elapsed between steps, as desired. As such, the time between performing each step may be 1 second or more, 10 seconds or more, 30 seconds or more, 60 seconds or more, 5 minutes or more, 10 minutes or more, 60 minutes or more and including 5 hours or more. In certain embodiments, each subsequent step is performed immediately after completion of the previous step. In other embodiments, a step may be performed after an incubation or waiting time after completion of the previous step, e.g., a few minutes to an overnight waiting time.

As used herein, the term “linker” or “linkage” refers to a linking moiety that connects two groups and has a backbone of 20 atoms or less in length. A linker or linkage may be a covalent bond that connects two groups or a chain of between 1 and 20 atoms in length, for example a chain of 1, 2, 3, 4, 5, 6, 8, 10, 12, 14, 16, 18 or 20 carbon atoms in length, where the linker may be linear, branched, cyclic or a single atom. In certain cases, one, two, three, four or five or more carbon atoms of a linker backbone may be optionally substituted with a sulfur, nitrogen or oxygen heteroatom. The bonds between backbone atoms may be saturated or unsaturated, and in some cases not more than one, two, or three unsaturated bonds are present in a linker backbone. The linker may include one or more substituent groups, for example with an alkyl, aryl or alkenyl group. A linker may include, without limitations, polyethylene glycol; ethers, thioethers, tertiary amines, alkyls, which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3 or 4 atoms, of the cyclic group are included in the backbone. A linker may be cleavable or non-cleavable.

As used herein, the term “alkyl” by itself or as part of another substituent refers to a saturated branched or straight-chain monovalent hydrocarbon radical derived by the removal of one hydrogen atom from a single carbon atom of a parent alkane. Alkyl groups of interest include, but are not limited to, methyl; ethyl, propyls such as propan-1-yl or propan-2-yl; and butyls such as butan-1-yl, butan-2-yl, 2-methyl-propan-1-yl or 2-methyl-propan-2-yl. In some embodiments, an alkyl group includes from 1 to 20 carbon atoms. In some embodiments, an alkyl group includes from 1 to 10 carbon atoms. In certain embodiments, an alkyl group includes from 1 to 6 carbon atoms, such as from 1 to 4 carbon atoms.

“Aryl” by itself or as part of another substituent refers to a monovalent aromatic hydrocarbon radical derived by the removal of one hydrogen atom from a single carbon atom of an aromatic ring system. Aryl groups of interest include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. In certain embodiments, an aryl group includes from 6 to 20 carbon atoms. In certain embodiments, an aryl group includes from 6 to 12 carbon atoms. Examples of an aryl group are phenyl and naphthyl.

“Heteroaryl” by itself or as part of another substituent, refers to a monovalent heteroaromatic radical derived by the removal of one hydrogen atom from a single atom of a heteroaromatic ring system. Heteroaryl groups of interest include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, triazole, benzotriazole, thiophene, triazole, xanthene, benzodioxole and the like. In certain embodiments, the heteroaryl group is from 5-20 membered heteroaryl. In certain embodiments, the heteroaryl group is from 5-10 membered heteroaryl. In certain embodiments, heteroaryl groups are those derived from thiophene, pyrrole, benzothiophene, benzofuran, indole, pyridine, quinoline, imidazole, oxazole and pyrazine.

“Substituted” refers to a group in which one or more hydrogen atoms are independently replaced with the same or different substituent(s). Substituents of interest include, but are not limited to, alkylenedioxy (such as methylenedioxy), -M, —R⁶⁰, —O⁻, ═O, —OR⁶⁰, —SR⁶⁰, —S⁻, ═S, —NR⁶⁰R⁶¹, ═NR⁶⁰, —CF₃, —CN, —OCN, —SCN, —NO, —NO₂, ═N₂, —N₃, —S(O)₂O⁻, —S(O)₂OH, —S(O)₂R⁶⁰, —OS(O)₂O⁻, —OS(O)₂R⁶⁰, —P(O)(O⁻)₂, —P(O)(OR⁶⁰) (O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —C(S) OR⁶⁰, —NR⁶²C(O)NR⁶⁰R⁶¹, —NR⁶²C(S)NR⁶²R⁶¹, —NR⁶²C(NR⁶³)NR⁶⁰R⁶¹ and —C(NR⁶²)NR⁶⁰R⁶¹ where M is halogen; R⁶⁰, R⁶¹, R⁶² and R⁶³ are independently hydrogen, alkyl, substituted alkyl, alkoxy, substituted alkoxy, cycloalkyl, substituted cycloalkyl, cycloheteroalkyl, substituted cycloheteroalkyl, aryl, substituted aryl, heteroaryl or substituted heteroaryl, or optionally R⁶⁰ and R⁶¹ together with the nitrogen atom to which they are bonded form a cycloheteroalkyl or substituted cycloheteroalkyl ring; and R⁶⁴ and R⁶⁵ are independently hydrogen, alkyl, substituted alkyl, aryl, cycloalkyl, substituted cycloalkyl, cycloheteroalkyl, substituted cycloheteroalkyl, aryl, substituted aryl, heteroaryl or substituted heteroaryl, or optionally R⁶⁴ and R⁶⁵ together with the nitrogen atom to which they are bonded form a cycloheteroalkyl or substituted cycloheteroalkyl ring. In certain embodiments, substituents include -M, —R⁶⁰, ═O, —OR⁶⁰, —SR⁶⁰, —S⁻, ═S⁻, —NR⁶⁰R⁶¹, ═NR⁶⁰, —CF₃, —CN, —OCN, —SCN, —NO, —NO₂, ═N₂, —N₃, —S(O)₂R⁶⁰, —OS(O)₂O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —NR⁶²C(O)NR⁶⁰R⁶¹. In certain embodiments, substituents include -M, —R⁶⁰, ═O, —OR⁶⁰, —SR⁶⁰, —NR⁶⁰R⁶¹, —CF₃, —CN, —NO₂, —S(O)₂R⁶⁰, —P(O)(OR⁶⁰)(O⁻), —OP(O)(OR⁶⁰)(OR⁶¹), —C(O)R⁶⁰, —C(O)OR⁶⁰, —C(O)NR⁶⁰R⁶¹, —C(O)O⁻. In certain embodiments, substituents include -M, —R⁶⁰, ═O, —OR⁶⁰, —SR⁶⁰, —NR⁶⁰R⁶¹, —CF₃, —CN, —NO₂, —S(O)₂R⁶⁰, —OP(O)(OR⁶⁰)(OR⁶¹), —C(O)R⁶⁰, —C(O)OR⁶⁰, —C(O)O⁻, where R⁶⁰, R⁶¹ and R⁶² are as defined above. For example, a substituted group may bear a methylenedioxy substituent or one, two, or three substituents selected from a halogen atom, a (1-4C)alkyl group and a (1-4C)alkoxy group. When the group being substituted is an aryl or heteroaryl group, the substituent(s) (e.g., as described herein) may be referred to as “aryl substituent(s)”.

Other definitions of terms may appear throughout the specification.

### DETAILED DESCRIPTION

Proteinaceous specific binding members that specifically bind to a polymeric dye are provided. Also provided are methods of using the specific binding members, e.g., in separating a polymeric dye-labeled cell from a sample, in analyte detection, etc., as described herein. Kits and systems for practicing the subject methods are also provided.

Before the various embodiments are described in greater detail, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

The section headings used herein are for organizational purposes only and are not to be construed as limiting the subject matter described in any way. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

The citation of any publication is for its disclosure prior to the filing date and should not be construed as an admission that the present claims are not entitled to antedate such publication by virtue of prior invention. Further, the dates of publication provided can be different from the actual publication dates which can be independently confirmed.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

All patents and publications, including all sequences disclosed within such patents and publications, referred to herein are expressly incorporated by reference.

In further describing the subject invention, a proteinaceous specific binding members that specifically bind to a polymeric dye are described first in greater detail. Next, methods of interest in which the subject specific binding members find use are reviewed. Systems and kits that may be used in practicing methods of the invention are also described.

### Specific Binding Members

As summarized above, the present disclosure provides specific binding members for polymeric dyes. As such, the specific binding members described herein specifically bind to a polymeric dye. In some embodiments, the specific binding member is proteinaceous.

As used herein, the term “specific binding member” refers to one member of a pair of molecules which have binding specificity for one another. One member of the pair of molecules may have an area on its surface, or a cavity, which specifically binds to an area on the surface of, or a cavity in, the other member of the pair of molecules. Thus the members of the pair have the property of binding specifically to each other. The present disclosure is concerned with specific binding members that include a proteinaceous member and a polymeric dye (e.g., as described herein) member, which specifically bind to each other. In some embodiments, the affinity between specific binding members in a binding complex is characterized by a K_{d} (dissociation constant) of 10⁻⁶ M or less, such as 10⁻⁷ M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, including 10⁻¹⁵ M or less. In some embodiments, the proteinaceous specific binding member specifically binds a polymeric dye of interest with high avidity. By high avidity is meant that the binding member specifically binds with an apparent affinity characterized by an apparent K_{d} of 10×10⁻⁹ M or less, such as 1×10⁻⁹ M or less, 3×10⁻¹⁰ M or less, 1×10⁻¹⁰ M or less, 3×10⁻¹¹ M or less, 1×10⁻¹¹ M or less, 3×10⁻¹² M or less or 1×10⁻¹² M or less.

As used herein, the term “proteinaceous” refers to a moiety that is composed of amino acid residues. A proteinaceous moiety may be a polypeptide.

In some embodiments, the proteinaceous specific binding member is an antibody molecule. The antibody molecule may be a whole antibody or an antibody fragment, e.g., a binding fragment of an antibody that specific binds to a polymeric dye. As used herein, the terms “antibody” and “antibody molecule” are used interchangeably and refer to a protein consisting of one or more polypeptides substantially encoded by all or part of the recognized immunoglobulin genes. The recognized immunoglobulin genes, for example in humans, include the kappa (k), lambda (l), and heavy chain genetic loci, which together comprise the myriad variable region genes, and the constant region genes mu (u), delta (d), gamma (g), sigma (e), and alpha (a) which encode the IgM, IgD, IgG, IgE, and IgA isotypes respectively. An immunoglobulin light or heavy chain variable region consists of a “framework” region (FR) interrupted by three hypervariable regions, also called “complementarity determining regions” or “CDRs”. The extent of the framework region and CDRs have been precisely defined (see, “Sequences of Proteins of Immunological Interest,” E. Kabat et al., U.S. Department of Health and Human Services, (1991)). The numbering of all antibody amino acid sequences discussed herein conforms to the Kabat system. The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen.

The term antibody is meant to include full length antibodies and antibody fragments, and may refer to a natural antibody from any organism, an engineered antibody, or an antibody generated recombinantly for experimental, therapeutic, or other purposes as further defined below. Antibody fragments are known in the art and include, but are not limited to, Fab, Fab′, F(ab′)2, Fv, scFv, or other antigen-binding subsequences of antibodies, either produced by the modification of whole antibodies or those synthesized de novo using recombinant DNA technologies. Antibodies may be monoclonal or polyclonal and may have other specific activities on cells (e.g., antagonists, agonists, neutralizing, inhibitory, or stimulatory antibodies). It is understood that the antibodies may have additional conservative amino acid substitutions which have substantially no effect on antigen binding or other antibody functions.

In certain embodiments, the specific binding member is an antibody, a Fab fragment, a F(ab′)₂ fragment, a scFv, a diabody or a triabody. In some cases, the specific binding member is a murine antibody or binding fragment thereof. In certain instances, the specific binding member is a recombinant antibody or binding fragment thereof.

In some embodiments, the proteinaceous specific binding member that specifically binds to a polymeric dye is support bound. As used herein, the terms “support bound” and “linked to a support” are used interchangeably and refer to a moiety (e.g., a specific binding member) that is linked covalently or non-covalently to a support of interest. Covalent linking may involve the chemical reaction of two compatible functional groups (e.g., two chemoselective functional groups, an electrophile and a nucleophile, etc.) to form a covalent bond between the two moieties of interest (e.g. a support and a specific binding member). In some cases, non-covalent linking may involve specific binding between two moieties of interest (e.g., two affinity moieties such as a hapten and an antibody or a biotin moiety and a streptavidin, etc.). In certain cases, non-covalent linking may involve absorption to a substrate.

Any convenient supports may be utilized in linking to the subject proteinaceous specific binding member. Supports of interest include, but are not limited to: solid substrates, where the substrate can have a variety of configurations, e.g., a sheet, bead, or other structure, such as a plate with wells; beads, polymers, particle, a fibrous mesh, hydrogels, porous matrix, a pin, a microarray surface, a chromatography support, and the like. In some instances, the support is selected from the group consisting of a particle, a planar solid substrate, a fibrous mesh, a hydrogel, a porous matrix, a pin, a microarray surface and a chromatography support. The support may be incorporated into a system that it provides for cell isolation assisted by any convenient methods, such as a manually-operated syringe, a centrifuge or an automated liquid handling system. In some cases, the support finds use in an automated liquid handling system for the high throughput isolation of cells, such as a flow cytometer.

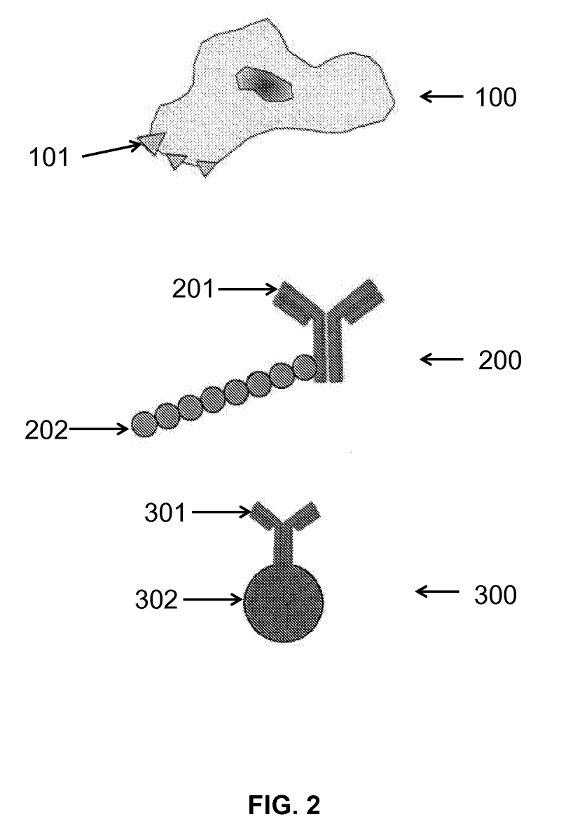

In certain instances, the support includes a magnetic particle. In some cases, the support is composed of colloidal magnetic particles. The term “particle” as used herein refers to a solid phase such as colloidal particles, microspheres, nanoparticles, or beads. Any convenient methods for generation of such particles may be used. In some instances, the particles are magnetic particles. The particles may be in a solution or suspension, or they may be in a lyophilized state prior to use. The lyophilized particle is then reconstituted in convenient buffer before contacting with the sample to be processed regarding the present invention. In some cases, the particle may have a size in diameter ranging from 100 nm to 1400 nm, such as from 200 to 500 nm. In certain instances, at least one specific binding member (e.g., as described herein) is coupled to the magnetic particle. FIG. 2 illustrates a support bound specific binding member (300) that includes a specific binding member (301) (e.g., an antibody that specifically binds a polymeric dye) linked to a magnetic particle (302).

As used herein, the term “magnetic” in “magnetic particle” refers to all subtypes of magnetic particles that may find use in methods of the invention, where examples of subtypes of magnetic particles that find use include, but are not limited to, ferromagnetic particles, superparamagnetic particles and paramagnetic particles. “Ferromagnetic” materials are strongly susceptible to magnetic fields and are capable of retaining magnetic properties when the field is removed. “Paramagnetic” materials have only a weak magnetic susceptibility and when the field is removed quickly lose their weak magnetism. “Superparamagnetic” materials are highly magnetically susceptible, i.e. they become strongly magnetic when placed in a magnetic field, but, like paramagnetic materials, rapidly lose their magnetism.

### Polymeric Dyes

As summarized above, the subject specific binding member specifically binds a polymeric dye. Polymeric dyes that may be specifically bound by a specific binding member of the invention are varied. In some instances, a polymeric dye is a multichromophore that has a structure capable of harvesting light to amplify the fluorescent output of a fluorophore. In some instances, the polymeric dye is capable of harvesting light and efficiently converting it to emitted light at a longer wavelength. In some cases, the polymeric dye has a light-harvesting multichromophore system that can efficiently transfer energy to nearby luminescent species (e.g., a “signaling chromophore”). Mechanisms for energy transfer include, for example, resonant energy transfer (e.g., Forster (or fluorescence) resonance energy transfer, FRET), quantum charge exchange (Dexter energy transfer) and the like. In some instances, these energy transfer mechanisms are relatively short range; that is, close proximity of the light harvesting multichromophore system to the signaling chromophore provides for efficient energy transfer. Under conditions for efficient energy transfer, amplification of the emission from the signaling chromophore occurs when the number of individual chromophores in the light harvesting multichromophore system is large; that is, the emission from the signaling chromophore is more intense when the incident light (the “pump light”) is at a wavelength which is absorbed by the light harvesting multichromophore system than when the signaling chromophore is directly excited by the pump light.

The multichromophore may be a conjugated polymer. Conjugated polymers (CPs) are characterized by a delocalized electronic structure and can be used as highly responsive optical reporters for chemical and biological targets. Because the effective conjugation length is substantially shorter than the length of the polymer chain, the backbone contains a large number of conjugated segments in close proximity. Thus, conjugated polymers are efficient for light harvesting and enable optical amplification via Forster energy transfer.

Polymeric dyes of interest include, but are not limited to, those dyes described by Gaylord et al. in US Publication Nos. 20040142344, 20080293164, 20080064042, 20100136702, 20110256549, 20120028828, 20120252986 and 20130190193 the disclosures of which are herein incorporated by reference in their entirety; and Gaylord et al., J. Am. Chem. Soc., 2001, 123 (26), pp 6417-6418; Feng et al., Chem. Soc. Rev., 2010, 39, 2411-2419; and Traina et al., J. Am. Chem. Soc., 2011, 133 (32), pp 12600-12607, the disclosures of which are herein incorporated by reference in their entirety.

In some embodiments, the polymeric dye includes a conjugated polymer including a plurality of first optically active units forming a conjugated system, having a first absorption wavelength (e.g., as described herein) at which the first optically active units absorbs light to form an excited state. The conjugated polymer (CP) may be polycationic, polyanionic and/or a charge-neutral conjugated polymer. The CPs may be water soluble for use in biological samples. Any convenient substituent groups may be included in the polymeric dyes to provide for increased water-solubility, such as a hydrophilic substituent group, e.g., a hydrophilic polymer, or a charged substituent group, e.g., groups that are positively or negatively charged in an aqueous solution, e.g., under physiological conditions.

The polymeric dye may have any convenient length. In some cases, the particular number of monomeric repeat units or segments of the polymeric dye may fall within the range of 2 to 500,000, such as 2 to 100,000, 2 to 30,000, 2 to 10,000, 2 to 3,000 or 2 to 1,000 units or segments, or such as 100 to 100,000, 200 to 100,000, or 500 to 50,000 units or segments.

The polymeric dyes may be of any convenient molecular weight (MW). In some cases, the MW of the polymeric dye may be expressed as an average molecular weight. the In some instances, the polymeric dye has an average molecular weight of from 500 to 500,000, such as from 1,000 to 100,000, from 2,000 to 100,000, from 10,000 to 100,000 or even an average molecular weight of from 50,000 to 100,000. In certain embodiments, the polymeric dye has an average molecular weight of 70,000.

In certain instances, the polymeric dye includes the following structure:

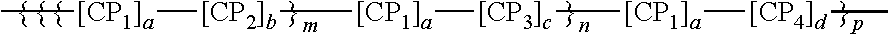

wherein CP₁, CP₂, CP₃ and CP₄ are independently a conjugated polymer segment or an oligomeric structure, wherein one or more of CP₁, CP₂, CP₃ and CP₄ are bandgap-lowering n-conjugated repeat units.

In some instances, the polymeric dye includes the following structure:

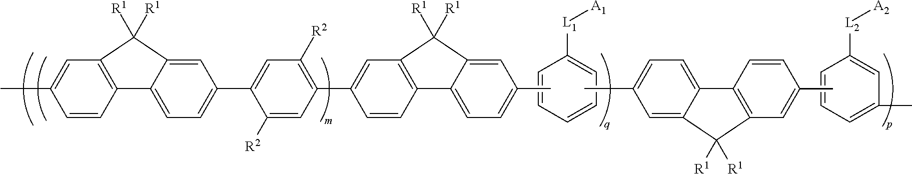

wherein each R¹ is independently a solubilizing group or a linker-dye; L₁ and L₂ are optional linkers; each R² is independently H or an aryl substituent; and each A₁ and A₂ is independently H or a fluorophore. Solubilizing groups of interest include alkyl, aryl and heterocycle groups further substituted with a hydrophilic group such as a polyethylglycol (e.g., a PEG of 2-20 units), a ammonium, a sulphonium, a phosphonium, and the like.

In some cases, the polymeric dye includes, as part of the polymeric backbone, one of the following structures:

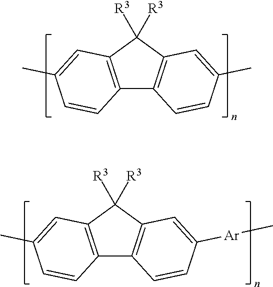

wherein each R³ is independently an optionally substituted alkyl or aryl group; Ar is an optionally substituted aryl or heteroaryl group; and n is 1 to 10000. In certain embodiments, R³ is an optionally substituted alkyl group. In certain embodiments, R³ is an optionally substituted aryl group. In some cases, R³ and or Ar is substituted with a polyethyleneglycol, a dye, a chemoselective functional group or a specific binding moiety.

In some instances, the polymeric dye includes the following structure:

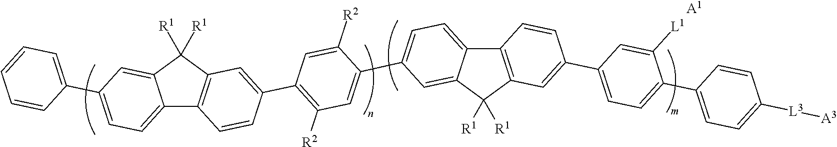

wherein: each R¹ is a solubilizing group or a linker-dye group; each R² is independently H or an aryl substituent; L₁ and L₂ are optional linkers; each A¹ and A³ are independently H, a fluorophore, a functional group or a specific binding moiety (e.g., an antibody); and n and m are each independently 0 to 10000, wherein n+m>1.

The subject polymeric dye may have one or more desirable spectroscopic properties, such as a particular absorption maximum wavelength, a particular emission maximum wavelength, extinction coefficient, quantum yield, and the like (see e.g., Chattopadhyay et al., “Brilliant violet fluorophores: A new class of ultrabright fluorescent compounds for immunofluorescence experiments.” Cytometry Part A, 81A(6), 456-466, 2012).

In some embodiments, the polymeric dye has an emission maximum wavelength ranging from 400 to 850 nm, such as 415 to 800 nm, where specific examples of emission maxima of interest include, but are not limited to: 421 nm, 510 nm, 570 nm, 602 nm, 650 nm, 711 nm and 786 nm. In certain embodiments, the polymeric dye has an emission maximum wavelength of 421 nm. In some instances, the polymeric dye has an emission maximum wavelength of 510 nm. In some cases, the polymeric dye has an emission maximum wavelength of 570 nm. In certain embodiments, the polymeric dye has an emission maximum wavelength of 602 nm. In some instances, the polymeric dye has an emission maximum wavelength of 650 nm. In certain cases, the polymeric dye has an emission maximum wavelength of 711 nm. In some embodiments, the polymeric dye has an emission maximum wavelength of 786 nm. In certain instances, the polymeric dye has an emission maximum wavelength of 421 nm±5 nm. In some embodiments, the polymeric dye has an emission maximum wavelength of 510 nm±5 nm. In certain instances, the polymeric dye has an emission maximum wavelength of 570 nm±5 nm. In some instances, the polymeric dye has an emission maximum wavelength of 602 nm±5 nm. In some embodiments, the polymeric dye has an emission maximum wavelength of 650 nm±5 nm. In certain instances, the polymeric dye has an emission maximum wavelength of 711 nm±5 nm. In some cases, the polymeric dye has an emission maximum wavelength of 786 nm±5 nm.

In some instances, the polymeric dye has an extinction coefficient of 1×10⁶ cm⁻¹M⁻¹ or more, such as 2×10⁶ cm⁻¹M⁻¹ or more, 2.5×10⁶ cm⁻¹M⁻¹ or more, 3×10⁶ cm⁻¹M⁻¹ or more, 4×10⁶ cm⁻¹M⁻¹ or more, 5×10⁶ cm⁻¹M⁻¹ or more, 6×10⁶ cm⁻¹M⁻¹ or more, 7×10⁶ cm⁻¹M⁻¹ or more, or 8×10⁶ cm⁻¹M⁻¹ or more. In certain embodiments, the polymeric dye and a quantum yield of 0.4 or more, such as 0.45 or more, 0.5 or more, 0.55 or more, 0.6 or more, 0.65 or more, 0.7 or more, or even more. In certain cases, the polymeric dye and a quantum yield of 0.5 or more.

In some cases, the specific binding member specifically binds the polymeric dye having an emission maximum of 421 nm. In some embodiments, the specific binding member specifically binds to the polymeric dye having an emission maximum of 421 nm and the polymeric dye having an emission maximum of 510 nm. In certain instances, the specific binding member binds the polymeric dye having an emission maximum of 421 nm with a specificity of 5:1 or more over the polymeric dye having an emission maximum of 510 nm, such as a specificity of 10:1 or more, 30:1 or more, 100:1 or more, or even more over the polymeric dye having an emission maximum of 510 nm.

In some instances, the specific binding member that binds the polymeric dye having an emission maximum of 421 nm has no cross-reactivity against the polymeric dye having an emission maximum of 510 nm. In some cases, by no cross-reactivity is meant that no specific binding of the specific binding member is detected in an in vitro binding assay.

In certain embodiments, the polymeric dye is a polymeric tandem dye. A specific binding member that specifically binds to a polymeric dye may also bind to a tandem dye, where the tandem dye includes that polymeric dye. Polymeric tandem dyes include two covalently linked dye moieties: a donor polymeric dye (e.g., as described herein) and an acceptor dye. A polymeric tandem dye may be excited at the excitation wavelength of the donor and may emit at the emission wavelength of the acceptor dye. Any convenient fluorophore may be utilized in the polymeric tandem dyes as an acceptor. Fluorophores of interest include, but are not limited to, fluorescent dyes such as fluorescein, 6-FAM, rhodamine, Texas Red, tetramethylrhodamine, carboxyrhodamine, carboxyrhodamine 6G, carboxyrhodol, carboxyrhodamine 110, Cascade Blue, Cascade Yellow, coumarin, Cy2, Cy3, Cy3.5, Cy5, Cy5.5, Cy-Chrome, phycoerythrin, PerCP (peridinin chlorophyll-a Protein), PerCP-Cy5.5, JOE (6-carboxy-4′,5′-dichloro-2′,7′-dimethoxyfluorescein), NED, ROX (5-(and-6)-carboxy-X-rhodamine), HEX, Lucifer Yellow, Marina Blue, Oregon Green 488, Oregon Green 500, Oregon Green 514, Alexa Fluor 350, Alexa Fluor 430, Alexa Fluor 488, Alexa Fluor 532, Alexa Fluor 546, Alexa Fluor 568, Alexa Fluor 594, Alexa Fluor 633, Alexa Fluor 647, Alexa Fluor 660, Alexa Fluor 680, Alexa Fluor 700, 7-amino-4-methylcoumarin-3-acetic acid, BODIPY FL, BODIPY FL-Br.sub.2, BODIPY 530/550, BODIPY 558/568, BODIPY 564/570, BODIPY 576/589, BODIPY 581/591, BODIPY 630/650, BODIPY 650/665, BODIPY R6G, BODIPY TMR, BODIPY TR, conjugates thereof, and combinations thereof. Lanthanide chelates of interest include, but are not limited to, europium chelates, terbium chelates and samarium chelates. In some embodiments, the polymeric tandem dye includes a polymeric dye linked to an acceptor fluorophore selected from the group consisting of Cy3, Cy3.5, Cy5, Cy5.5, Cy7, Alexa488, Alexa 647 and Alexa700.

The subject proteinaceous specific binding member may bind to any convenient epitope of the target polymeric dye, the backbone, substituents, e.g., solubilizing groups, linker dyes, etc., and the like. The subject proteinaceous specific binding member may be prepared using any convenient method. In some embodiments, the proteinaceous specific binding member that specifically binds a polymeric dye is an antibody that is prepared using any convenient method, where the polymeric dye is used as an immunogen, by itself or conjugated to an immunogenic carrier, such as KLH, pre-S HBsAg, other viral or eukaryotic proteins, or the like.

### Cells and Polynucleotides

Aspects of the disclosure provide nucleic acids encoding proteinaceous specific binding members. For recombinant production of the proteinaceous specific binding members, the nucleic acid encoding the specific binding member is inserted into a replicable vector for further cloning (amplification of the DNA) or for expression. DNA encoding the specific binding member is readily isolated and sequenced using any convenient procedures (e.g., by using oligonucleotide probes that are capable of binding specifically to genes encoding the specific binding member). Any convenient vectors may be utilized. The vector components may include, but are not limited to, one or more of the following: a signal sequence, an origin of replication, one or more marker genes, an enhancer element, a promoter, and a transcription termination sequence.

As such, the disclosure provides an isolated polynucleotide (i.e., nucleic acid) encoding a proteinaceous specific binding member that specifically binds a polymeric dye (e.g., as described herein). As used herein the term “isolated,” when used in the context of an isolated polynucleotide, refers to a polynucleotide of interest that is at least 60% free, at least 75% free, at least 90% free, at least 95% free, at least 98% free, and even at least 99% free from other components with which the polynucleotide is associated with prior to purification. In some embodiments, the polynucleotide is recombinant. In some cases, the polynucleotide is a cDNA.

Also provided are cells expressing a proteinaceous specific binding member that specifically binds a polymeric dye. In some cases, the cells express an antibody, or antibody fragment. In some instances, the cell is derived from an immunized mouse. In certain embodiments, the cell is a hybridoma. In certain cases, the cell is recombinantly produced. The proteinaceous specific binding member prepared from the cells can be purified using, for example, hydroxylapatite chromatography, gel electrophoresis, dialysis, and affinity chromatography, with affinity chromatography being a purification technique of interest. In some cases, the specific binding member is an antibody and the suitability of protein A as an affinity ligand depends on the species and isotype of any immunoglobulin Fc domain that is present in the antibody. Protein A can be used to purify antibodies that are based on human γ1, γ2, or γ4 heavy chains. Protein G may be used for human γ3. The matrix to which the affinity ligand is attached may in some cases be agarose, but in other cases other matrices are available. Mechanically stable matrices such as controlled pore glass or poly(styrenedivinyl)benzene allow for faster flow rates and shorter processing times than can be achieved with agarose. Where the antibody includes a CH₃ domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered. Following any preliminary purification step(s), the mixture including the specific binding member of interest and contaminants may be subjected to low pH hydrophobic interaction chromatography using an elution buffer at a pH between 2.5 and 4.5, in some cases performed at low salt concentrations (e.g., from 0-0.25M salt).

### Methods

As summarized above, aspects of the invention include methods using the polymeric dye specific binding members. Polymeric dye specific binding members, e.g., as described herein, find use in a variety of different applications. Applications of interest include, but are not limited to: separation applications, analyte detection applications, etc. These types of different applications are now review further in greater detail.

### Separation Applications

Aspects of the invention include methods of separating a polymeric dye-labeled cell from a sample. In some embodiments, the methods include: contacting a sample including a polymeric dye-labeled cell with a support bound proteinaceous specific binding member that specifically binds to the polymeric dye of the polymeric dye-labeled cell; separating the support from the sample; and eluting the polymeric dye-labeled cell from the support using a biocompatible aqueous eluent.

Any convenient method may be used to contact the sample with a support bound proteinaceous specific binding member that specifically binds to the polymeric dye of the polymeric dye-labeled cell. In some instances, the sample is contacted with the support bound proteinaceous specific binding member under conditions in which the specific binding member specifically binds to the polymeric dye-labeled cell, if present.

For specific binding of the proteinaceous specific binding member with the polymeric dye-labeled cell, an appropriate solution may be used that maintains the viability of the cells. The solution may be a balanced salt solution, e.g., normal saline, PBS, Hank's balanced salt solution, etc., conveniently supplemented with fetal calf serum, human platelet lysate or other factors, in conjunction with an acceptable buffer at low concentration, such as from 5-25 mM. Convenient buffers include HEPES, phosphate buffers, lactate buffers, etc. Various media are commercially available and may be used according to the nature of the target cells, including dMEM, HBSS, dPBS, RPMI, Iscove's medium, etc., frequently supplemented with fetal calf serum or human platelet lysate. The final components of the solution may be selected depending on the components of the cell sample which are included.

The sample may include a heterogeneous cell population from which target cells are isolated. In some instances, the sample includes peripheral whole blood, peripheral whole blood in which erythrocytes have been lysed prior to cell isolation, cord blood, bone marrow, density gradient-purified peripheral blood mononuclear cells or homogenized tissue. In some cases, the sample includes hematopoetic progenitor cells (e.g., CD34+ cells) in whole blood, bone marrow or cord blood. In certain embodiments, the sample includes tumor cells in peripheral blood. In certain instances, the sample is a sample including (or suspected of including) viral cells (e.g., HIV).

The temperature at which specific binding of the proteinaceous specific binding member to the polymeric dye-labeled cell takes place may vary, and in some instances may range from 5° C. to 50° C., such as from 10° C. to 40° C., 15° C. to 40° C., 20° C. to 40° C., e.g., 20° C., 25° C., 30° C., 35° C. or 37° C. (e.g., as described above). In some instances, the temperature at which specific binding takes place is selected to be compatible with the viability of the polymeric dye-labeled cell and/or the biological activity of the proteinaceous specific binding member. In certain instances, the temperature is 25° C., 30° C., 35° C. or 37° C. In certain cases, the proteinaceous specific binding member is an antibody or fragment thereof and the temperature at which specific binding takes place is room temperature (e.g., 25° C.), 30° C., 35° C. or 37° C. Any convenient incubation time for specific binding may be selected to allow for the formation of a desirable amount of binding complex, and in some instances, may be 1 minute (min) or more, such as 2 min or more, 10 min or more, 30 min or more, 1 hour or more, 2 hours or more, or even 6 hours or more.

Any convenient method may be used to prepare a polymeric dye-labeled cell. In some cases, the polymeric dye-labeled cell includes a polymeric dye covalently linked to a target cell of interest. In certain embodiments, the polymeric dye-labeled cell includes a target cell specifically bound to a polymeric dye-labeled affinity agent. In certain cases, the sample may be pre-treated by contacting a sample containing (or suspected of containing) a target cell of interest with a polymeric dye-labeled affinity agent under conditions in which the affinity agent specifically binds with the target cell to produce a polymeric dye-labeled cell. As such, in some embodiments, the method further includes contacting the target cell with the polymeric dye-labeled affinity agent to produce the polymeric dye-labeled cell. The contacting may be achieved using any convenient means. In some cases, a concentrated aliquot of a polymeric dye-labeled affinity agent is added to a sample including target cell(s) under conditions sufficient for the affinity agent to specifically bind to the target cell. Excess affinity agent may then be removed or separated from the labeled cells.

For specific binding of the affinity agent to the target cell, an appropriate solution may be used that maintains the viability of the cells. The solution may be a balanced salt solution, e.g., normal saline, PBS, Hank's balanced salt solution, etc., conveniently supplemented with fetal calf serum, human platelet lysate or other factors, in conjunction with an acceptable buffer at low concentration, such as from 5-25 mM (e.g., as described above).

The temperature at which specific binding of the affinity agent and the target cell takes place may vary, and in some instances may range from 5° C. to 50° C., such as from 10° C. to 40° C., 15° C. to 40° C., 20° C. to 40° C., e.g., 20° C., 25° C., 30° C., 35° C. or 37° C. (e.g., as described above). Any convenient incubation time for specific binding may be selected to allow for the formation of a desirable amount of binding complex (e.g., as described above).

The polymeric dye-labeled affinity agent may be a conjugate of the polymeric dye (e.g., as described herein) and an affinity agent that specifically binds the target cell of interest. Any convenient affinity agents may be utilized in the conjugate. Affinity agents of interest include, but are not limited to, those affinity agents that specifically bind cell surface proteins of a variety of cell types, including but not limited to, stem cells, T cells, dendritic cells, B Cells, granulocytes, leukemia cells, lymphoma cells, NK cells, macrophages, monocytes, fibroblasts, epithelial cells, endothelial cells and erythroid cells.

As used herein the terms “affinity agent” and “capture agent” are used interchangeably and refer to an agent that binds an analyte through an interaction that is sufficient to permit the agent to extract and concentrate the analyte from a homogeneous mixture of different analytes. The binding interaction may be mediated by an affinity region of the capture agent. In some cases, capture agents include antibodies, which are well known in the art. Capture agents may “specifically bind” to one or more analytes. Thus, the term “capture agent” refers to a molecule or a multi-molecular complex which can specifically bind an analyte, e.g., specifically bind an analyte for the capture agent with a dissociation constant (K_{D}) of 10⁻⁶ or less without binding to other targets, such as 10⁻⁷ M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, including 10⁻¹⁵ M or less.

FIG. 2 illustrates a polymeric dye labeled affinity agent (200) that is composed of an affinity agent (e.g., an antibody, 201), which specifically binds the lineage-specific cell marker (101) of a target cell (100), conjugated to a polymeric dye (202).

Antibody-polymeric dye conjugates find use in the subject methods, e.g., for labeling a target cell, particle, target or analyte with a polymeric dye. For example, antibody conjugates find use in labeling cells to be processed (e.g., detected, analyzed, and/or sorted) in a flow cytometer. The conjugates may include antibodies that specifically bind to, e.g., cell surface proteins of a variety of cell types (e.g., as described herein). The labeled antibody conjugates may be used to investigate a variety of biological (e.g., cellular) properties or processes such as cell cycle, cell proliferation, cell differentiation, DNA repair, T cell signaling, apoptosis, cell surface protein expression and/or presentation, and so forth. Antibody conjugates may be used in any application that includes (or may include) antibody-mediated labeling of a cell, particle or analyte. Conjugates that find use in the subject methods optionally include a linker between the dye and the affinity agent.

In some embodiments, the sample including (or suspected of including) a target cell is contacted with a polymeric dye-labeled affinity agent under conditions in which the affinity agent specifically binds the target cell, if present, to produce a polymeric dye-labeled cell. In certain embodiments, the sample includes a polymeric dye-labeled cell.

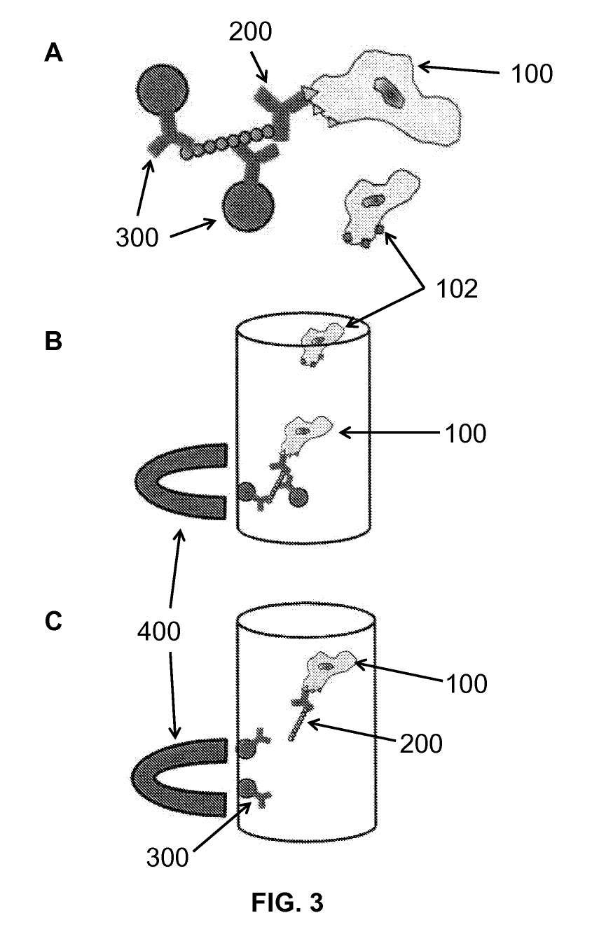

FIG. 3A illustrates the labeling of a target cell (100) with a polymeric dye labeled affinity agent (200) (e.g., a lineage specific antibody conjugated to a polymeric dye) and capturing of the target cell with a support-bound proteinaceous specific binding member (300) (e.g., a magnetic particle bound anti-polymeric dye antibody).

A variety of cells may be targeted for separation using the subject methods. Target cells of interest include, but are not limited to, stem cells, e.g., pluripotent stem cells, hematopoietic stem cells, T cells, T regulator cells, dendritic cells, B Cells, e.g., memory B cells, antigen specific B cells, granulocytes, leukemia cells, lymphoma cells, virus cells (e.g., HIV cells) NK cells, macrophages, monocytes, fibroblasts, epithelial cells, endothelial cells, and erythroid cells. Target cells of interest include cells that have a convenient cell surface marker or antigen that may be captured by a convenient affinity agent or conjugates thereof. In some embodiments, the target cell is selected from HIV containing cell, a Treg cell, an antigen-specific T-cell populations, tumor cells or hematopoetic progenitor cells (CD34+) from whole blood, bone marrow or cord blood. Any convenient cell surface proteins or cell markers may be targeted for specific binding to polymeric dye-labeled affinity agents in the subject methods. In some embodiments, the target cell includes a cell surface marker selected from a cell receptor and a cell surface antigen. FIG. 2 illustrates a schematic of a target cell (100) that includes a cell lineage-specific marker (101) on the surface of the target cell. For example, the target cell may include a cell surface antigen such as CD11b, CD123, CD14, CD15, CD16, CD19, CD193, CD2, CD25, CD27, CD3, CD335, CD36, CD4, CD43, CD45RO, CD56, CD61, CD7, CD8, CD34, CD1c, CD23, CD304, CD235a, T cell receptor alpha/beta, T cell receptor gamma/delta, CD253, CD95, CD20, CD105, CD117, CD120b, Notch4, Lgr5 (N-Terminal), SSEA-3, TRA-1-60 Antigen, Disialoganglioside GD2 and CD71.

The support-bound proteinaceous specific binding member includes a proteinaceous specific binding member (e.g., as described herein) linked to a support (e.g., as described herein), where the linkage may be covalent or non-covalent. In some cases, the term “support bound” refers to a covalent linkage to the surface of a solid support. Use of a support bound specific binding member provides for immobilization and/or separation of any target cell to which the specific binding member binds. A variety of methods may be utilized to separate a target cell from a sample via immobilization on a support.

In some embodiments of the method, the separating step includes applying an external magnetic field to immobilize a magnetic particle. Any convenient magnet may be used as a source of the external magnetic field (e.g., magnetic field gradient). In some cases, the external magnetic field is generated by a magnetic source, e.g. by a permanent magnet or electromagnet. In some cases, immobilizing the magnetic particles means the magnetic particles accumulate near the surface closest to the magnetic field gradient source, i.e. the magnet.

The separating may further include one or more optional washing steps to remove unbound material of the sample from the support. Any convenient washing methods may be used, e.g., washing the immobilized support with a biocompatible buffer which preserves the specific binding interaction of the polymeric dye and the specific binding member. Separation and optional washing of unbound material of the sample from the support provides for an enriched population of target cells where undesired cells and material may be removed.

FIGS. 3A and B illustrate the capturing of the target cell (100) with a polymeric dye labelled affinity agent conjugate (200) and a support-bound proteinaceous specific binding member (300) (e.g., a magnetic particle bound anti-polymeric dye antibody) and the application of the external magnetic field of a magnet (400) to retain magnetic particle bound cells (100). Non-binding cells (102) which do not include lineage-specific markers are washed away from the immobilized target cells.

Aspects of the subject methods include eluting the target cells from the support with a biocompatible aqueous eluent that disassociates the polymeric dye-specific binding member complex under conditions in which target cell viability and activity is preserved. As such, the disclosure provides a biocompatible aqueous eluent for eluting the polymeric dye-labeled cell from the support. Any convenient methods may be used to elute the cell (e.g., the polymeric dye-labeled cell) from the support whereby the cell remains viable.

FIG. 3C illustrates the release of target cells (100) from the magnetic particles immobilized using the external magnetic field of a magnet (400), using a biocompatible elution buffer to produce purified and isolated cells which may include the polymeric dye labeled affinity agent (200).

As used herein, the term “biocompatible” refers to an aqueous eluent that is non-cytotoxic and non-denaturing to the target cell. In addition, the components of the biocompatible aqueous eluent may be selected such that the eluent has no adverse effects on subsequent analysis and/or use of the target cells. In some embodiments, the biocompatible aqueous eluent includes a binding competitor or inhibitor of the polymeric dye-specific binding member complex that is capable of disrupting the specific binding of the polymeric dye and the binding member. By disrupting the specific binding is meant that the two binding members may be more easily disassociated. The binding competitor or inhibitor may have any convenient affinity for one of the binding members. In some cases, the binding competitor or inhibitor binds with a relatively low affinity, but may disrupt specific binding at a sufficient and desirable concentration in the eluent. It is understood that the biocompatible aqueous eluent may further include a variety of components in conjunction with the binding competitor or inhibitor to promote dissociation of a viable polymeric dye labeled cell.

In some cases, the binding competitor or inhibitor is itself a polymer. Any convenient polymer(s) may be utilized in the subject biocompatible aqueous eluents. Polymers of interest include, but are not limited to, polyethylene glycols, polypeptides, oligonucleotides, polyvinyl alcohols, polyacrylamide, polydecylmethacrylate, polystyrene, dendrimer molecule, polycaprolactone (PCL), polylactic acid (PLA), poly(lactic-co-glycolic acid) (PLGA), polyglycolic acid (PGA), polyhydroxybutyrate (PHB), and the like. In some instances, the polymer selected for inclusion in the biocompatible aqueous eluent has a backbone structural feature that is similar to the polymeric dye. In certain cases, the polymer selected for inclusion in the biocompatible aqueous eluent has a sidechain structural feature that is similar to the polymeric dye. In certain instances, the polymer selected for inclusion in the biocompatible aqueous eluent binds non-specifically to the specific binding member.

In some embodiments, the biocompatible aqueous eluent includes a polymer that competitively binds to the proteinaceous specific binding member. In certain instances, the biocompatible aqueous eluent includes a polyalkylene oxide, such as a polyethylene glycol. As used herein, a “polyethylene glycol” or “PEG” refers to a polymer including a chain described by the formula —(CH₂—CH₂—O—)ₙ— or a derivative thereof. In some embodiments, “n” is 5000 or less, such as 1000 or less, 500 or less, 200 or less, 100 or less or even 50 or less. It is understood that the PEG polymer may be of any convenient length and may include a variety of terminal groups, including but not limited to, alkyl, aryl, hydroxyl, amino, acyl, acyloxy, and amido terminal groups.

As such, in certain embodiments, the biocompatible aqueous eluent is non-proteinaceous, i.e., the eluent includes no proteinaceous components. In some cases, the biocompatible aqueous eluent is non-proteinaceous, is capable of disrupting the specific binding of the polymeric dye and the binding member and has no adverse effects on subsequent analysis and/or use of the target cells.

As such, the subject methods of separation produce a sample including an enriched or purified population of target cells from which the support has been removed, which may facilitate the detection and/or analysis of the cell. In some cases, the target cell may also be separated from other components of the method, such as the polymeric dye and/or the affinity agent. The subject methods and immobilized specific binding members may be used to selectively deplete a subset of cell types from a mixed population through use of polymeric dye-labelled affinity agents which selectively bind to the subset.

In certain embodiments, the method further includes detecting the target cell (e.g., a polymeric dye-labeled cell). In certain embodiments, the method further includes analyzing the polymeric dye-labeled cell. In some instances, the method further includes flow cytometrically analyzing the polymeric dye-labeled cell.

Detecting the cell in a flow cytometer may include exciting a fluorescent dye with one or more lasers at an interrogation point of the flow cytometer, and subsequently detecting fluorescence emission from the dye using one or more optical detectors. It may be desirable, in addition to detecting the particle, to determine the number of particles (e.g., cells) separated, or utilizing one or components of the methods (e.g., polymeric dye-labeled affinity agent) for the purpose of sorting the particles. Accordingly, in some embodiments, the methods further include counting, sorting, or counting and sorting the labeled particle (e.g., target cell).

In detecting, counting and/or sorting particles, a liquid medium including the particles is first introduced into the flow path of the flow cytometer. When in the flow path, the particles are passed substantially one at a time through one or more sensing regions (e.g., an interrogation point), where each of the particles is exposed individually to a source of light at a single wavelength and measurements of light scatter parameters and/or fluorescent emissions as desired (e.g., two or more light scatter parameters and measurements of one or more fluorescent emissions) are separately recorded for each particle. The data recorded for each particle is analyzed in real time or stored in a data storage and analysis means, such as a computer, as desired. U.S. Pat. No. 4,284,412 describes the configuration and use of a flow cytometer of interest equipped with a single light source while U.S. Pat. No. 4,727,020 describes the configuration and use of a flow cytometer equipped with two light sources. Flow cytometers having more than two light sources may also be employed.

More specifically, in a flow cytometer, the particles are passed, in suspension, substantially one at a time in a flow path through one or more sensing regions (or “interrogation points”) where in each region each particle is illuminated by an energy source. The energy source may include an illuminator that emits light of a single wavelength, such as that provided by a laser (e.g., He/Ne or argon) or a mercury arc lamp with appropriate filters. For example, light at 488 nm may be used as a wavelength of emission in a flow cytometer having a single sensing region. For flow cytometers that emit light at two distinct wavelengths, additional wavelengths of emission light may be employed, where specific wavelengths of interest include, but are not limited to: 535 nm, 635 nm, and the like.

In series with a sensing region, detectors, e.g., light collectors, such as photomultiplier tubes (or “PMT”), are used to record light that passes through each particle (in certain cases referred to as forward light scatter), light that is reflected orthogonal to the direction of the flow of the particles through the sensing region (in some cases referred to as orthogonal or side light scatter) and fluorescent light emitted from the particles, if it is labeled with fluorescent marker(s), as the particle passes through the sensing region and is illuminated by the energy source. Each of forward light scatter (or FSC), orthogonal light scatter (SSC), and fluorescence emissions (FL1, FL2, etc.) comprise a separate parameter for each particle (or each “event”). Thus, for example, two, three or four parameters can be collected (and recorded) from a particle labeled with two different fluorescence markers.

Accordingly, in flow cytometrically assaying the particles, the particles may be detected and uniquely identified by exposing the particles to excitation light and measuring the fluorescence of each particle in one or more detection channels, as desired. The excitation light may be from one or more light sources and may be either narrow or broadband. Examples of excitation light sources include lasers, light emitting diodes, and arc lamps. Fluorescence emitted in detection channels used to identify the particles and binding complexes associated therewith may be measured following excitation with a single light source, or may be measured separately following excitation with distinct light sources. If separate excitation light sources are used to excite the particle labels, the labels may be selected such that all the labels are excitable by each of the excitation light sources used.

Flow cytometers further include data acquisition, analysis and recording means, such as a computer, wherein multiple data channels record data from each detector for the light scatter and fluorescence emitted by each particle as it passes through the sensing region. The purpose of the analysis system is to classify and count particles wherein each particle presents itself as a set of digitized parameter values. In flow cytometrically assaying (e.g., detecting, counting and/or sorting) particles in methods of the invention, the flow cytometer may be set to trigger on a selected parameter in order to distinguish the particles of interest from background and noise. “Trigger” refers to a preset threshold for detection of a parameter and may be used as a means for detecting passage of a particle through the laser beam. Detection of an event that exceeds the threshold for the selected parameter triggers acquisition of light scatter and fluorescence data for the particle. Data is not acquired for particles or other components in the medium being assayed which cause a response below the threshold. The trigger parameter may be the detection of forward scattered light caused by passage of a particle through the light beam. The flow cytometer then detects and collects the light scatter and fluorescence data for the particle.

A particular subpopulation of interest is then further analyzed by “gating” based on the data collected for the entire population. To select an appropriate gate, the data is plotted so as to obtain the best separation of subpopulations possible. This procedure may be performed by plotting forward light scatter (FSC) vs. side (i.e., orthogonal) light scatter (SSC) on a two dimensional dot plot. The flow cytometer operator then selects the desired subpopulation of particles (i.e., those cells within the gate) and excludes particles that are not within the gate. Where desired, the operator may select the gate by drawing a line around the desired subpopulation using a cursor on a computer screen. Only those particles within the gate are then further analyzed by plotting the other parameters for these particles, such as fluorescence.

Flow cytometric analysis of the particles, as described above, yields qualitative and quantitative information about the particles. Where desired, the above analysis yields counts of the particles of interest in the sample. As such, the above flow cytometric analysis protocol provides data regarding the numbers of one or more different types of particles in a sample.

Also provided is a method of determining whether a cell is present in a sample. In some cases, the method includes: contacting a sample suspected of including a polymeric dye-labeled cell with a support bound proteinaceous specific binding member that specifically binds to the polymeric dye of the polymeric dye-labeled cell; separating the support from the sample; subjecting the support to elution conditions including a biocompatible aqueous elution buffer to produce an eluent; and evaluating whether the isolated polymeric dye-labeled cell is present in the eluent to determine whether a cell is present in a sample.

In some instances, the method further includes, prior to the contacting, combining a sample suspected of including a target cell with a polymeric dye-specific binding member conjugate. In certain embodiments of the method, the evaluating includes flow cytometrically analyzing the eluent (e.g., as described herein). In some embodiments of the method, the support bound proteinaceous specific binding member includes a support that is a magnetic particle and the separating includes applying an external magnetic field.

Also provided is a method of analyzing a target cell. In some instances, the method includes: contacting a sample including a polymeric dye-labeled cell with a support bound proteinaceous specific binding member that specifically binds to the polymeric dye of the polymeric dye-labeled cell; separating the support from the sample; dissociating the polymeric dye-labeled cell from the support using a biocompatible aqueous elution buffer to produce an eluent including the dissociated cell; and flow cytometrically analyzing the dissociated cell. In some embodiments of the method, the method further includes, prior to the contacting, combining a sample including a target cell with a polymeric dye-specific binding member conjugate to produce the polymeric dye-labeled cell. In certain embodiments of the method, the proteinaceous specific binding member is linked to a support that includes a magnetic particle and the separating includes applying an external magnetic field.

Also provided are methods of separating a polymeric dye-labeled target from a sample. The subject proteinaceous specific binding members that specifically bind a polymeric dye find use in a variety of methods of separation, detection and/or analysis. Any convenient methods and assay formats where pairs of specific binding members such as avidin-biotin or hapten-anti-hapten antibodies find use, are of interest. Methods and assay formats of interest that may be adapted for use with the subject compositions include, but are not limited to, flow cytometry methods, in-situ hybridization methods, enzyme-linked immunosorbent assays (ELISAs), western blot analysis, magnetic cell separation assays and fluorochrome purification chromatography.

As such, any convenient targets may be utilized in the methods. Targets of interest include, but are not limited to, a nucleic acid, such as an RNA, DNA, PNA, CNA, HNA, LNA or ANA molecule, a protein, such as a fusion protein, a modified protein, such as a phosphorylated, glycosylated, ubiquitinated, SUMOylated, or acetylated protein, or an antibody, a peptide, an aggregated biomolecule, a cell (e.g., as described herein), a small molecule, a vitamin and a drug molecule. As used herein, the term “a target protein” refers to all members of the target family, and fragments thereof. The target protein may be any protein of interest, such as a therapeutic or diagnostic target, including but not limited to: hormones, growth factors, receptors, enzymes, cytokines, osteoinductive factors, colony stimulating factors and immunoglobulins. The term “target protein” is intended to include recombinant and synthetic molecules, which can be prepared using any convenient recombinant expression methods or using any convenient synthetic methods, or purchased commercially.

In some embodiments, the method includes: (a) contacting a sample including a polymeric dye-labeled target with a proteinaceous specific binding member (e.g., as described herein) that specifically binds to the polymeric dye of the polymeric dye-labeled target to form a complex; and (b) separating the complex from the sample.

In some instances, the method further includes detecting and/or analyzing the polymeric dye-labeled target. Any convenient methods may be utilized to detect and/or analyse the polymeric dye-labeled target in conjunction with the subject methods and compositions. Methods of analyzing a target of interest that find use in the subject methods, include but are not limited to, flow cytometry, in-situ hybridization, enzyme-linked immunosorbent assays (ELISAs), western blot analysis, magnetic cell separation assays and fluorochrome purification chromatography. Detection methods of interest include but are not limited to fluorescence spectroscopy, nucleic acid sequencing, fluorescence in-situ hybridization (FISH), protein mass spectroscopy, flow cytometry, Detection may be achieved directly via a reporter molecule, or indirectly by a secondary detection system. The latter may be based on any one or a combination of several different principles including but not limited to, antibody labelled anti-species antibody and other forms of immunological or non-immunological bridging and signal amplification systems (e.g., biotin-streptavidin technology, protein-A and protein-G mediated technology, or nucleic acid probe/anti-nucleic acid probes, and the like). The label used for direct or indirect detection may be any detectable reported molecule. Suitable reporter molecules may be those known in the field of immunocytochemistry, molecular biology, light, fluorescence, and electron microscopy, cell immunophenotyping, cell sorting, flow cytometry, cell visualization, detection, enumeration, and/or signal output quantification. Labels of interest include, but are not limited to fluorophores, luminescent labels, metal complexes, radioisotopes, biotin, streptavidin, enzymes, or other detection labels and combination of labels such as enzymes and a luminogenic substrate. Enzymes of interest and their substrates include alkaline phosphatase, horseradish peroxidase, beta-galactosidase, and luciferase, and the like. More than one antibody of specific and/or non-specific nature might be labelled and used simultaneously or sequentially to enhance target detection, identification, and/or analysis. Labels of interest include, but are not limited to FITC (fluorescein isothiocyanate) AMCA (7-amino-4-methylcoumarin-3-acetic acid), Alexa Fluor 488, Alexa Fluor 594, Alexa Fluor 350, DyLight350, phycoerythrin, allophycocyanin and stains for detecting nuclei such as Hoechst 33342, LDS751, TO-PRO and DAPI.

Any convenient method may be used to prepare a polymeric dye-labeled target. The sample may be pre-treated by contacting a sample containing (or suspected of containing) a target of interest with a polymeric dye-labeled affinity agent under conditions in which the affinity agent specifically binds with the target to produce a polymeric dye-labeled cell. As such, the polymeric dye-labeled target may include a target specifically bound to a polymeric dye-labeled affinity agent. In some cases the polymeric dye may be covalently bound to the target. Any convenient means for covalently labelling a target with a polymeric dye, including but not limited to those methods and reagents described by Hermanson, Bioconjugate Techniques, Third edition, Academic Press, 2013. In certain instances, the proteinaceous specific binding member is support bound (e.g., as described above).

### Analyte Detection Applications

Aspects of the invention include methods of detecting an analyte in a sample using polymeric dye specific binding members, e.g., as described above, where the polymeric dye specific binding members may be part of a signal producing system, e.g., that further includes a polymeric dye, e.g., as described above. Contacting the sample with a polymeric dye specific binding member may result in labeling of an analyte of interest, e.g., one that has been tagged with a first analyte specific binding member that includes a polymeric dye, and provide for detection of the analyte, e.g., by fluorescence. In some instances, the analyte is labeled via complexation with members of a signal producing system, which include a polymeric dye labeled analyte specific binding member and a polymeric dye specific binding member.

In some embodiments, the method includes contacting the sample with a signal producing system that includes a polymeric dye specific binding member under conditions in which the members of the signal producing system form a complex with the analyte. In certain embodiments, the contacting step occurs under conditions sufficient for a member of the signal producing system, e.g., the analyte specific polymeric dye labeled binding member, to specifically bind the analyte. In some cases, the signal producing system includes a first reagent that includes a polymeric dye labeled specific binding moiety that specifically binds the analyte, and a second reagent that includes a polymeric dyes specific binding member, which second reagent may include a variety of different types of labels, including directly detectable and indirectly detectable labels. As used herein, the term “detection reagent” refers to any molecule that is used to facilitate optical detection of an analyte.

As used herein, the terms “analyte” and “target” are used interchangeably and refer to any substance to be analyzed, detected, measured, or labeled. Analytes of interest include, but are not limited to, proteins, peptides, hormones, haptens, antigens, antibodies, receptors, enzymes, nucleic acids, polysaccarides, chemicals, polymers, pathogens, toxins, organic drugs, inorganic drugs, cells, tissues, microorganisms, viruses, bacteria, fungi, algae, parasites, allergens, pollutants, and combinations thereof. By convention, where cells of a given cell type are to be detected, either the cellular component molecules or the cell itself can be described as an analyte.

Signal producing systems in which the polymeric dye specific binding members of the invention find use include energy transfer systems, e.g., where the polymeric dye specific binding member is labeled with an acceptor moiety that is configured to receive light emitted by a polymeric dye component of the signal producing system. The acceptor moiety that labels the polymeric dye specific binding member (e.g., by being stably associated therewith, such as covalently bound thereto) may vary, as desired. Acceptor moieties of interest include those that are configured to receive energy from the polymeric dye (which may be viewed as the donor) and produce a signal in response thereto which is distinct from that produced by the polymeric dye. Acceptor moieties that may be employed include protein or non-proteinaceous acceptor moieties. Examples of acceptor moities that are protein include, but are not limited to, green fluorescent protein (GFP), blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz, GFPuv, destabilised EGFP (dEGFP), destabilised ECFP (dECFP), destabilised EYFP (dEYFP), HcRed, t-HcRed, DsRed, DsRed2, t-dimer2, t-dimer2(12), mRFP1, pocilloporin, Renilla GFP, Monster GFP, paGFP, Kaede protein or a Phycobiliprotein, or a biologically active variant or fragment of any one thereof. Examples of acceptor moieties that are not proteins include, but are not limited to, Alexa Fluor dye, Bodipy dye, Cy dye, fluorescein, dansyl, umbelliferone, fluorescent microsphere, luminescent microsphere, fluorescent nanocrystal, Marina Blue, Cascade Blue, Cascade Yellow, Pacific Blue, Oregon Green, Tetramethylrhodamine, Rhodamine, Texas Red, rare earth element chelates, or any combination or derivatives thereof. Acceptor moieties of interest also include fluorescent nanocrystal. Nanocrystals, or “quantum dots”, have several advantages over organic molecules as fluorescent labels, including resistance to photodegradation, improved brightness, non-toxicity, and size dependent, narrow emission spectra that enables the monitoring of several processes simultaneously. Additionally, the absorption spectrum of nanocrystals is continuous above the first peak, enabling all sizes, and hence all colors, to be excited with a single excitation wavelength. Fluorescent nanocrystals may be attached, or “bioconjugated”, to proteins in a variety of ways. For example, the surface cap of a “quantum dot” may be negatively charged with carboxylate groups from either dihydrolipoic acid (DHLA) or an amphiphilic polymer. Proteins can be conjugated to the DHLA-nanocrystals electrostatically, either directly or via a bridge consisting of a positively charged leucine zipper peptide fused to recombinant protein. The latter binds to a primary antibody with specificity for the intended target. Alternatively, antibodies, streptavidin, or other proteins are coupled covalently to the polyacrylate cap of the nanocrystal with conventional carbodiimide chemistry. Also of interest as acceptor moieties are fluorescent microspheres. These are typically made from polymers, and contain fluorescent molecules (for example fluorescein GFP or YFP) incorporated into the polymer matrix, which can be conjugated to a variety of reagents. Fluorescent microspheres may be labelled internally or on the surface. Internal labelling produces very bright and stable particles with typically narrow fluorescent emission spectra. With internal labelling, surface groups remain available for conjugating ligands (for example, proteins) to the surface of the bead. Internally-labelled beads are used extensively in imaging applications, as they display a greater resistance to photobleaching. Also of interest as acceptor moieties are quenchers which receive emitted light from the polymeric dye but do not produce a signal in response thereto.

Single producing systems in which polymeric dye specific binding members also include those which are configured to amplify an initial signal, e.g., where the polymeric dye specific binding member includes a tag (which may be viewed as an indirectly detectable label) that is a specific binding member pair in which the other member is labeled. For example, the polymeric dye specific binding may be labeled with a first binding member pair of the sets of pairs listed in Table 1, below, where the second member of the pair is then further labeled with a label, which may be directly or indirectly detectable.

TABLE 1

| Antigen | Antibody |
|---|---|
| Biotin | Avidin, streptavidin, or anti-biotin Antibody |
| IgG (an immunoglobulin) | protein A or protein G |
| Drug | Drug receptor |
| Toxin | Toxin receptor |
| Carbohydrate | Lectin or carbohydrate receptor |
| Peptide | Peptide receptor |
| Nucleotide | Complimentary nucleotide |
| Protein | Protein receptor |
| Enzyme substrate | Enzyme |
| Nucleic acid | Nucleic acid |
| Hormone | Hormone receptor |
| Psoralen | Nucleic acid |
| Target molecule | RNA or DNA aptamer |

Any convenient protocol for contacting the sample with the signal producing system that includes the polymeric dye specific binding member may be employed. The particular protocol that is employed may vary, e.g., depending on whether the sample is in vitro or in vivo, and whether a dye compound or dye conjugate is used. For in vitro protocols, contact of the sample with the dye compound or dye conjugate may be achieved using any convenient protocol. In some instances, the sample includes cells which are maintained in a suitable culture medium, and the dye compound or dye conjugate is introduced into the culture medium. For in vivo protocols, any convenient administration protocol may be employed. Depending upon the target, the response desired, the manner of administration, e.g. i.v. s.c. i.p. oral, etc, the half-life, the number of cells present, various protocols may be employed. The term “sample” as used herein relates to a material or mixture of materials, typically, although not necessarily, in fluid form, containing one or more components of interest (e.g., an analyte).

### Systems

Aspects of the invention further include systems for use in practicing the subject methods. A sample analysis system may include a flow channel loaded with a sample including a labeled cell. The labeled cell may include a polymeric dye-specific binding member conjugate specifically bound to a target cell (e.g., as described herein).

In some embodiments, the system is a flow cytometric system including: a flow cytometer including a flow path; a composition in the flow path, wherein the composition includes: a cell-containing biological sample; a polymeric dye-specific binding member conjugate that specifically binds a target cell; and a support bound proteinaceous specific binding member that specifically binds to the polymeric dye.

In certain embodiments, the sample includes a polymeric dye-labeled cell including the polymeric dye-specific binding member conjugate specifically bound to a target cell.

In some cases, the support bound proteinaceous specific binding member includes a support that is a magnetic particle. As such, in certain instances, the system may also include a controllable external paramagnetic field configured for application to an assay region of the flow channel.

In some instances of the system, the polymeric dye includes a conjugated polymer including a plurality of first optically active units forming a conjugated system, having a first absorption wavelength at which the first optically active units absorbs light to form an excited state. In some cases of the system, the polymeric dye has an emission maximum selected from 421 nm, 510 nm, 570 nm, 602 nm, 650 nm, 711 nm and 786 nm (e.g., an emission maximum of 421 nm or 510 nm). In certain instances of the system, the polymeric dye is a polymeric tandem dye.

In certain aspects, the system may also include a light source configured to direct light to an assay region of the flow channel. The system may include a detector configured to receive a signal from an assay region of the flow channel, wherein the signal is provided by the fluorescent composition. Optionally further, the sample analysis system may include one or more additional detectors and/or light sources for the detection of one or more additional signals.

In certain aspects, the system may further include computer-based systems configured to detect the presence of the fluorescent signal. A “computer-based system” refers to the hardware means, software means, and data storage means used to analyze the information of the present invention. The minimum hardware of the computer-based systems of the present invention includes a central processing unit (CPU), input means, output means, and data storage means. A skilled artisan can readily appreciate that any one of the currently available computer-based system are suitable for use in the present invention. The data storage means may include any manufacture including a recording of the present information as described above, or a memory access means that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g., word processing text file, database format, etc.

A “processor” references any hardware and/or software combination that will perform the functions required of it. For example, any processor herein may be a programmable digital microprocessor such as available in the form of an electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

In addition to the sensor device and signal processing module, e.g., as described above, systems of the invention may include a number of additional components, such as data output devices, e.g., monitors and/or speakers, data input devices, e.g., interface ports, keyboards, etc., fluid handling components, power sources, etc.

In certain aspects, the system includes a flow cytometer. Suitable flow cytometry systems and methods for analyzing samples include, but are not limited to those described in Ormerod (ed.), Flow Cytometry: A Practical Approach, Oxford Univ. Press (1997); Jaroszeski et al. (eds.), Flow Cytometry Protocols, Methods in Molecular Biology No. 91, Humana Press (1997); Practical Flow Cytometry, 3rd ed., Wiley-Liss (1995); Virgo, et al. (2012) Ann Clin Biochem. January; 49(pt 1):17-28; Linden, et. al., Semin Throm Hemost. 2004 October; 30(5):502-11; Alison, et al. J Pathol, 2010 December; 222(4):335-344; and Herbig, et al. (2007) Crit Rev Ther Drug Carrier Syst. 24(3):203-255; the disclosures of which are incorporated herein by reference. In certain instances, flow cytometry systems of interest include BD Biosciences FACSCanto™ flow cytometer, BD Biosciences FACSVantage™, BD Biosciences FACSort™, BD Biosciences FACSCount™, BD Biosciences FACScan™, and BD Biosciences FACSCalibur™ systems, a BD Biosciences Influx™ cell sorter, BD Biosciences Jazz™ cell sorter and BD Biosciences Aria™ cell sorter or the like.

In certain embodiments, the subject systems are flow cytometer systems which incorporate one or more components of the flow cytometers described in U.S. Pat. Nos. 3,960,449; 4,347,935; 4,667,830; 4,704,891; 4,770,992; 5,030,002; 5,040,890; 5,047,321; 5,245,318; 5,317,162; 5,464,581; 5,483,469; 5,602,039; 5,620,842; 5,627,040; 5,643,796; 5,700,692; 6,372,506; 6,809,804; 6,813,017; 6,821,740; 7,129,505; 7,201,875; 7,544,326; 8,140,300; 8,233,146; 8,753,573; 8,975,595; 9,092,034; 9,095,494 and 9,097,640; the disclosures of which are herein incorporated by reference.

Other systems may find use in practicing the subject methods. In certain aspects, the system may be a fluorimeter or microscope loaded with a sample having a fluorescent composition of any of the embodiments discussed herein. The fluorimeter or microscope may include a light source configured to direct light to the assay region of the flow channel. The fluorimeter or microscope may also include a detector configured to receive a signal from an assay region of the flow channel, wherein the signal is provided by the fluorescent composition.

### Compositions

Aspects of the invention further include compositions for use in practicing the subject methods. The compositions of the invention can be provided for use in, for example, the methodologies described above.

In some embodiments, the composition includes a polymeric dye-labeled cell (e.g., as described herein); and a support bound proteinaceous specific binding member (e.g., as described herein) that specifically binds to the polymeric dye of the polymeric dye-labeled cell (e.g., as described herein).

Also provided is a composition including: a polymeric dye-labeled antibody (e.g., as described herein) that specifically binds a target cell (e.g., as described herein); and a support bound proteinaceous specific binding member that specifically binds to the polymeric dye (e.g., as described herein).

Also provided is a composition including: a cell-containing biological sample; a polymeric dye-specific binding member conjugate that specifically binds a target cell; and a support bound proteinaceous specific binding member that specifically binds to the polymeric dye.

In certain embodiments of the composition, the support bound proteinaceous specific binding member includes a support selected from the group consisting of a particle, a solid substrate, a fibrous mesh, a hydrogel, a porous matrix, a pin, a microarray surface and a chromatography support. In certain instances, the support includes a magnetic particle.

In some instances of the composition, the polymeric dye includes a conjugated polymer including a plurality of first optically active units forming a conjugated system, having a first absorption wavelength at which the first optically active units absorbs light to form an excited state. In some cases of the composition, the polymeric dye has an emission maximum selected from 421 nm, 510 nm, 570 nm, 602 nm, 650 nm, 711 nm and 786 nm (e.g., an emission maximum of 421 nm or 510 nm). In certain instances of the composition, the polymeric dye is a polymeric tandem dye.

### Kits

Aspects of the invention further include kits for use in practicing the subject methods and compositions. The compositions of the invention can be included as reagents in kits either as starting materials or provided for use in, for example, the methodologies described above.

A kit may include a support bound proteinaceous specific binding member (e.g., as described herein) that specifically binds to a polymeric dye; and one or more components selected from a polymeric dye, a polymeric tandem dye, a polymeric dye-specific binding member conjugate, a cell, a support, an biocompatible aqueous elution buffer, and instructions for use.

In certain embodiments, the kit finds use in the isolation of particle-free specific cell subpopulations from anti-coagulated whole blood, such as in the absence of additional sample processing or red blood cell lysis, for subsequent flow cytometric analysis or cell culture. As such, in some instances, the kit includes one or more components suitable for treating whole blood, such as one or more anticoagulants. Anticoagulants of interest include, but are not limited to, heparin, coumarins, factor Xa inhibitors, thrombin inhibitors, and derivatives thereof.

The one or more additional components may be provided in separate containers (e.g., separate tubes, bottles, or wells in a multi-well strip or plate).

In certain aspects, the kit may further include reagents for performing a flow cytometric assay. Examples of said reagents include buffers for at least one of reconstitution and dilution of the first and second detectible molecules, buffers for contacting a cell sample with one or both of the first and second detectible molecules, wash buffers, control cells, control beads, fluorescent beads for flow cytometer calibration and combinations thereof. The kit may also include one or more cell fixing reagents such as paraformaldehyde, glutaraldehyde, methanol, acetone, formalin, or any combinations or buffers thereof. Further, the kit may include a cell permeabilizing reagent, such as methanol, acetone or a detergent (e.g., triton, NP-40, saponin, tween 20, digitonin, leucoperm, or any combinations or buffers thereof. Other protein transport inhibitors, cell fixing reagents and cell permeabilizing reagents familiar to the skilled artisan are within the scope of the subject kits.

The composition may be provided in a liquid composition, such as any suitable buffer. Alternatively, the composition may be provided in a dry composition (e.g., may be lyophilized), and the kit may optionally include one or more buffers for reconstituting the dry composition. In certain aspects, the kit may include aliquots of the fluorescent composition provided in separate containers (e.g., separate tubes, bottles, or wells in a multi-well strip or plate).

In addition, one or more components may be combined into a single container, e.g., a glass or plastic vial, tube or bottle. In certain instances, the kit may further include a container (e.g., such as a box, a bag, an insulated container, a bottle, tube, etc.) in which all of the components (and their separate containers) are present. The kit may further include packaging that is separate from or attached to the kit container and upon which is printed information about the kit, the components of the and/or instructions for use of the kit.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, DVD, portable flash drive, etc., on which the information has been recorded. Yet another means that may be present is a website address which may be used via the Internet to access the information at a removed site. Any convenient means may be present in the kits.

### Utility

The compositions, system and methods as described herein may find use in a variety of applications, including diagnostic and research applications, in which the separation, detection and/or analysis of a analyte of interest (e.g., a cell) is desirable.

Such applications include methodologies such as cytometry, microscopy, immunoassays (e.g. competitive or non-competitive), assessment of a free analyte, assessment of receptor bound ligand, and so forth. The compositions, system and methods described herein may be useful in analysis of any of a number of samples, including but not limited to biological fluids, cell culture samples, and tissue samples. In certain aspects, the compositions, system and methods described herein may find use in methods where analytes are detected in a sample using fluorescent labels, such as in fluorescent activated cell sorting or analysis, immunoassays, immunostaining, and the like.

In some cases, the methods and compositions find use in any assay format where the separation detection and/or analysis of a target from a sample is of interest, including but not limited to, flow cytometry, in-situ hybridization, enzyme-linked immunosorbent assays (ELISAs), western blot analysis, magnetic cell separation assays and fluorochrome purification chromatography. The subject compositions may be adapted for use in any convenient applications where pairs of specific binding members find use, such as biotin-streptavidin and hapten-anti-hapten antibody.

In some instances, the methods and compositions find use in the isolation of particle-free specific cell subpopulations from anti-coagulated whole blood, in the absence of additional sample processing or red blood cell lysis, for subsequent flow cytometric analysis or cell culture. In certain instances, the methods and compositions find use in the enrichment of antigen-specific T-cell populations through use of polymeric dye-conjugated streptavidin multimers. In some embodiments, the methods and compositions find use in the isolation of regulatory (Treg) cells from peripheral blood prior to expansion and reinjection into patients for cell therapy. In certain cases, the methods and compositions find use in the enrichment of particle-free specific cell populations prior to nucleic acid analysis to facilitate the diagnosis of viral infection, including HIV. In some instances, the methods and compositions find use in the selective depletion of several specific cell populations from a heterogeneous mixture to yield an enriched cell population, free of any additional bound antibody. In certain cases, the methods and compositions find use in high throughput cell isolation using an automated liquid-handling system. In some embodiments, the methods and compositions find use in the isolation of circulating tumor cells in peripheral blood to monitor metastases. In certain instances, the methods and compositions find use in the isolation of hematopoetic progenitor cells (CD34+) from whole blood, bone marrow or cord blood for cell therapy, regenerative medicine and tissue engineering applications. In some cases, applications include the reversible immunoprecipitation of soluble protein analytes. In certain cases, applications include the reversible capture and elution of analytes of interest, including but not limited to, proteins, nucleic acids, viruses and bacteria.

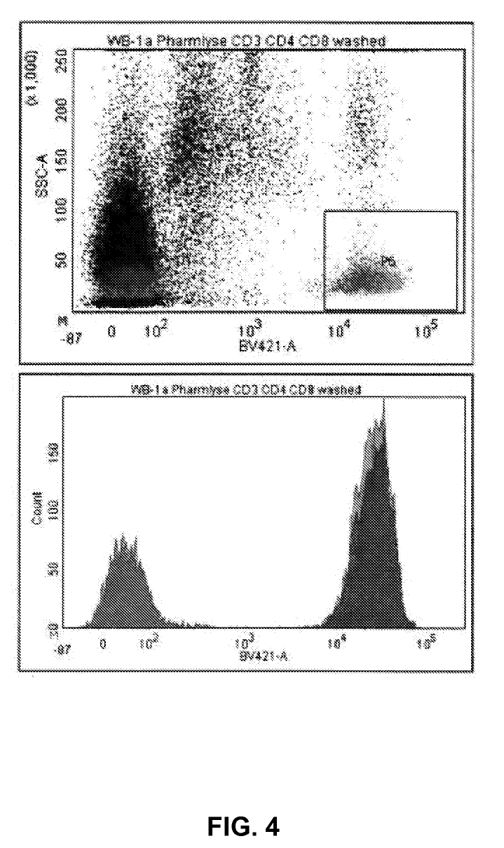

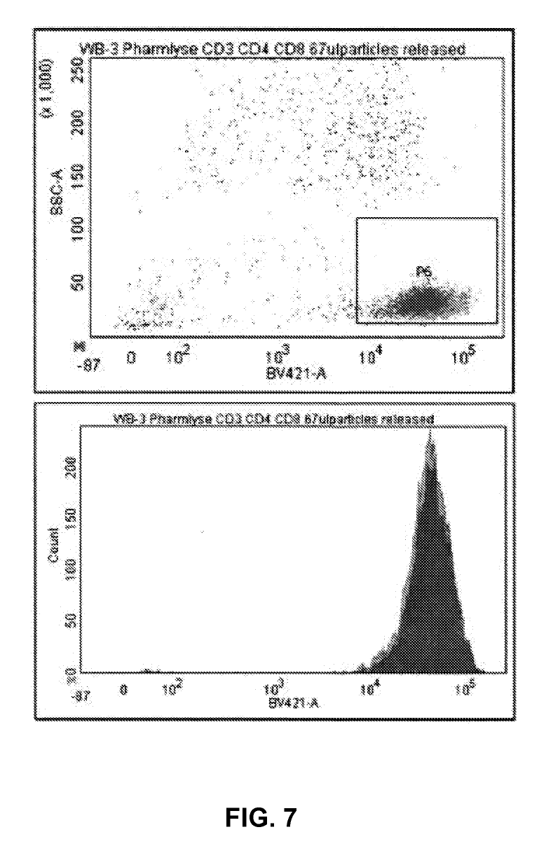

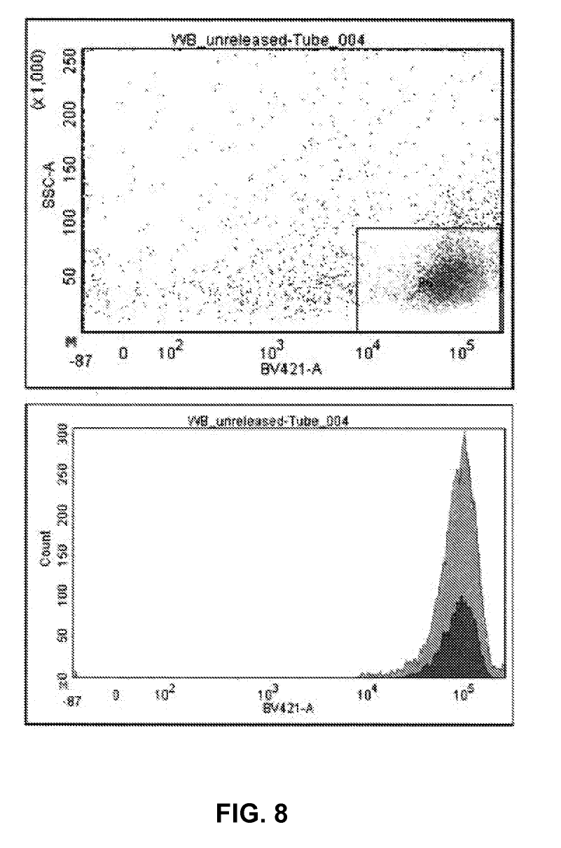

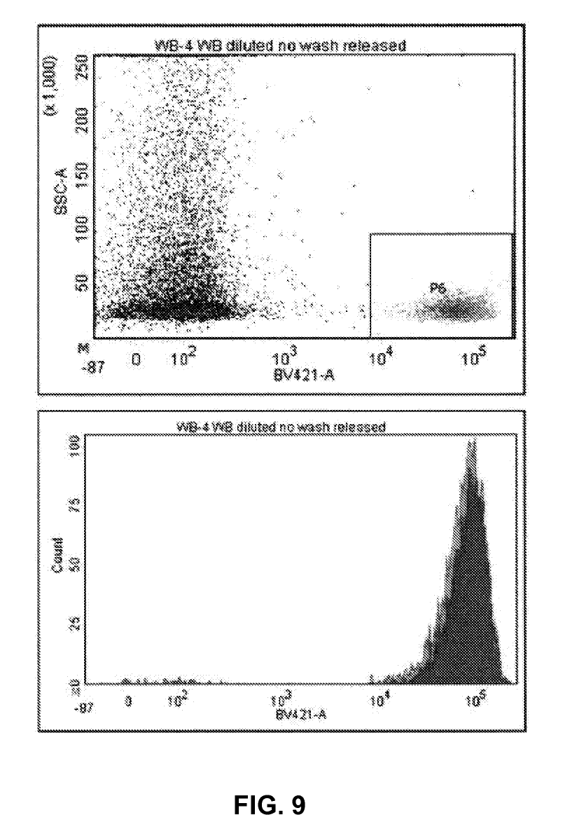

The methods and compositions described herein may find use in any application where purified target cells that retain their natural cell viability and bioactivity are desired. For example, FIGS. 4-9 illustrate that specific cell subtypes may be separated from a sample via specific binding to magnetic particles and subsequently released to produce purified cell samples.

The following examples are offered by way of illustration and not by way of limitation.

### EXPERIMENTAL

### Example 1: Preparation of Antibody to Polymeric Dye

### Immunogen:

BV 421 was conjugated to KLH and BSA using standard thiol/maleimide coupling chemistry.

### Immunization:

Balb/c mice were then immunized via both the subcutaneous (s.c.) and intraperitoneal (i.p.) routes with 50 μg of BV421-KLH emulsified in Complete Freund's Adjuvant (CFA). Mice were subsequently boosted (i.p.) 3× at two-week intervals with 50 μg of the same antigen in Incomplete Freund's Adjuvant (IFA). Immune response was determined by ELISA analysis after the fourth immunization. The two mice with the highest serum anti-BV421 titer as determined by ELISA were selected to undergo the Hybridoma Fusion process.

### Spleen Harvesting and Splenocyte Collection:

Mice were sacrificed and spleens were harvested. Spleens were then processed to single cell suspensions using the plunger mashing method.

### Hybridoma/Fusion:

FO myeloma cells were used to fuse collected splenocytes using hybridoma fusion methods. Two different fusions were performed, one for each of the selected animals.

### ELISA Screening Plate Preparation:

BSA-BV421 was coated at 2 μg/ml dilution with PBS on Nunc's Maxisorp 96-well plates and incubated overnight. Plates were then washed 5× in an automated plate washer using a combination of PBS and Tween and then blocked using BD's Elispot Assay Diluent for one hour. Plates were then tapped dry and stored in a −20° C. freezer for use in primary fusion, 1^{st} Subclone and 2^{nd} Subclone screens.

A similar process was performed when coating plates with the BV421 tandems (BV605, BV650, BV711, BV786) and BV510 (BV=Brilliant Violet™) with the only difference being that the dyes were not conjugated to BSA. These plates were used for cross-reactivity analysis when performing clone selection.

### ELISA Screening:

### Primary Fusion Screen:

Ten days post fusion, each of the fusions plated into 96-well plates had 100 μL of tissue culture supernatant transferred from each well to the BSA-BV421 coated ELISA plates (12 plates/fusion). Supernatants were left to incubate on the plates for one hour at room temperature, and then aspirated and washed in an automated plate washer with 200 μL of wash solution (PBS and Tween) five times with a final aspiration cycle. 100 μL of secondary antibody solution (Anti-mouse IgG Subclass specific HRP) was added to each well by use of an automated liquid handling platform and left to incubate for one hour. Plates were washed again with the method described above and 100 μL of ABTS substrate solution (0.1% of Hydrogen Peroxide) was added to each well. The plates were incubated for an additional 30 minutes at room temperature and then absorbance (optical density at 405 nm) was read of each well for each plate in a spectrophotometer. Wells that had an OD higher than three times the background (OD>0.3) were considered to be positive. Selected wells were then analyzed via microscopy to confirm if viable cell colonies were present in each selected site. All positive wells that contained cells in them were then transferred to 24-well plates for expansion and subsequent testing for cross-reactivity, flow cytometry and isotyping.

### Cross-reactive Analysis:

Three days after the primary fusion clone selection, supernatant from each of the potential clones from both fusions were tested for cross-reactivity with other BV dyes. A total of 12 clones (from both fusions) were found to be specific for the polymeric dyes, thus binding BV421, the tandem dyes and BV510. Three additional clones were found to bind BV421 and tandem dyes built off of the BV421 structure specifically, and thus not cross-reacting with BV510. A plate coated with Phycoerythrin (PE) was used as a negative control. Any clones that bound to the BV dyes and PE were discarded as non-specific antibody producers.

1^{st} and 2^{nd} Subclone Screening by ELISA:

The screening of clones by ELISA follows the same protocol as described at the Primary Fusion Screen stage.

### Flow Cytometry Screening:

Strategy: The screening system for flow cytometry was designed to serve three purposes: 1) It was meant to be simple and efficient by making use of a mouse cell line that can be grown in large cell numbers to serve as a screening tool of large numbers of clones. 2) It was also designed to screen for clones that recognize BV421 in solution. To address this requirement we looked for an antibody conjugated to BV421 that recognized a marker widely expressed on the cell surface. 3) The BV421 conjugated antibody is raised to a host other than mouse. Using an antibody raised in a species other than mouse eliminated the possibility of non-specific binding of the second step reagent to the antibody that carried the BV421 polymer.

The system used for screening met all three requirements. It utilized the 2D6 cell line for screening that can be expanded in culture and fixed in methanol for storage until the fusion screening is complete. This cell line is a mouse Th1 cell line therefore expresses surface CD3 molecules. A hamster anti-CD3
BV421 antibody clone 145-2C11 for surface CD3 binding and expression of free floating BV 421 was selected.

### Protocol:

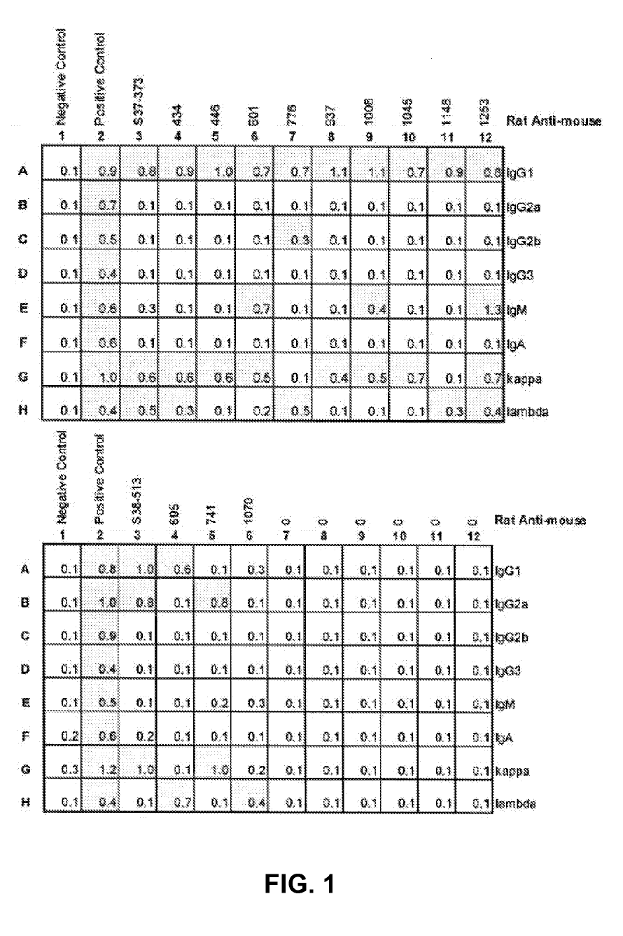

2D6 cells were stained with hamster anti-mouse CD3 BV421 (0.125 μg/l0⁶ cells) in 96-well U-bottom microtiter plates at RT for 30 min. Cells were then washed 2× with staining buffer (1×PBS, 2% FBS, 0.09% sodium azide). The cells were subsequently stained with hybridoma supernatants derived from the BV 421 fusion (50 μL neat supernatant per/well) for 45 min at RT. The cells were next washed 2× with staining buffer. Specific binding of supernatants to BV 421 was determined using a goat anti-mouse IgG-PE antibody (hamster adsorbed) at 0.25 μg per 10⁶ cells for 45-60 min at RT. Cells were then washed twice and data was acquired in a BD FACS Canto II. Data were analyzed using FlowJo V9.6 software (FIG. 1).

### Process:

Hybridoma supernatants screened positive for BV421 binding via ELISA were subsequently screened for reactivity to cell-anchored BV 421 via an antibody by flow according to the strategy described above. Hybridoma supernatants were screened by flow at primary, first and second subclone screening. The table below summarizes the results following flow screening and the number of clones that were moved forward during the screening process.

### Rationale for Picking Clones for Further Subcloning:

Clones that worked in ELISA and flow cytometry (using the strategy described above) with “clean” isotype (single isotype) moved on to 1^{st} and subsequent 2^{nd} subcloning.

### Isotype Analysis:

Clones that were selected for their reactivity against BV 421 were tested for their isotype via ELISA after primary fusion screen, 1st subclone and 2nd subclone screens. (See FIG. 1).

Isotype test results for clones selected from fusions S37 and S38 at Primary Fusion Screen: Ten clones selected S37 and four clones from S38 were tested for their isotype. This analysis determines the monoclonality of the clones selected, and if such is present, the isotype of the antibodies produced by the cells. A mixed isotype may signal the presence of more than one clone in a specific well.

### Results

TABLE 1
Summary of the clone selection process from primary fusion screening to
second subcloning based on ELISA, flow cytometry and isotyping results.

| | | | | #moved |
|---|---|---|---|---|
| | | Flow positive | # | to large |
| | Elisa positive wells | wells | subcloned | scale |
| Primary | 34 | 16 | 14 | N/A |
| screening | | | | |
| First | 569 (from 12 parent | 36 subclones | 9 | N/A |
| subcloning | clones - two clones | from 12 parent | | |
| | were lost) | clones | | |
| Second | 440 (from 10 | 40 subclones | N/A | 2 |
| subcloning | subclones - one | from 10 parent | | |
| | subclone was lost) | clones | | |

The majority of the clones developed against BV421 were isotyped as IgG, λ. In general, hybridoma fusions yield clones that produce antibodies with the IgG,K isotype and finding lambda light chains is considered a rarity.

### Example 2: Magnetic Separation of Particle Bound Cells

The general flow cytometry assay described herein is adapted for the magnetic separation of particle bound cells.

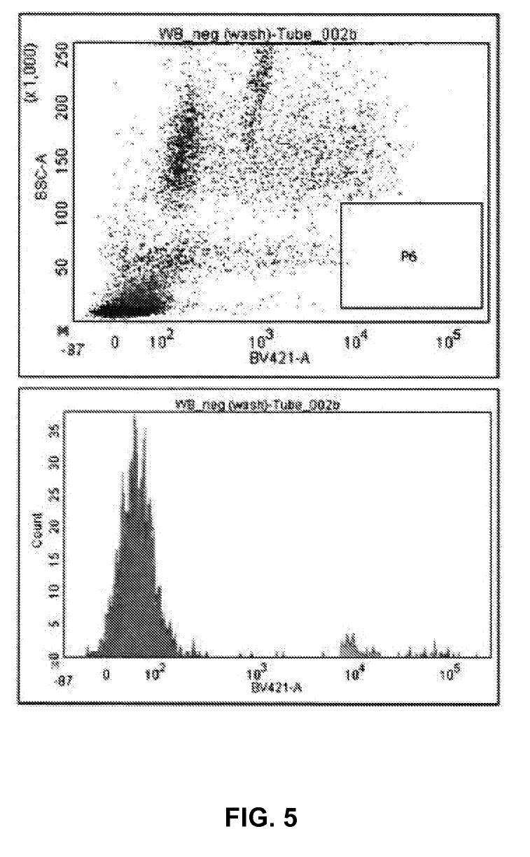

FIGS. 4 and 5 provide data illustrating both negative and positive selection of specific cell subtypes and release of particle-bound cells. Peripheral blood mononuclear cells were stained with an anti CD3-BV421 conjugate followed by red blood cell lysis. The sample was contacted with anti-BV421 (clone 537-937.75.32) modified magnetic particles. Magnetically-labeled components were isolated using a magnet and the bound and unbound cell fractions analyzed by flow cytometry. The magnetic particle-bound cells were subsequently treated with release buffer and exposed to a magnet to remove the liberated particles and yield a purified particle-free cell population.

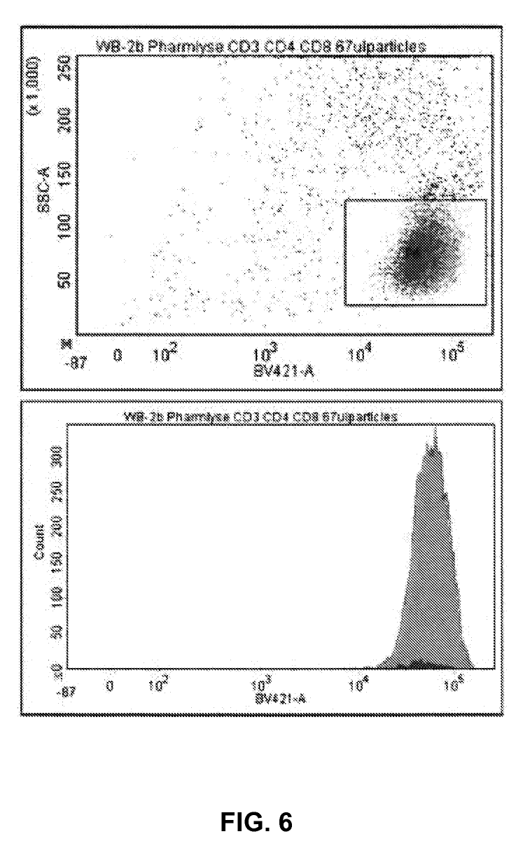

FIG. 6 illustrates the purification of particle-free lymphocyte subpopulations from whole blood without additional lysis or centrifuge based intermediate cell washing. Anticoagulated human whole blood was stained with anti CD3 BV421, followed by the addition of anti BV421-modified magnetic particles. The sample was subjected to magnetic separation of particle-bound cells. The cells were washed on the magnet and subsequently released using release buffer and analyzed by flow cytometry (note; some scattering events from contaminating platelets were observed, but should not affect the purity of the CD3 positive mononuclear cell population). Such a workflow is highly amenable to high throughput automation and sample processing for clinical analysis.

### Example 3: Energy Transfer

### A. Reagent Preparation

BV421-labeled anti CD4 (clone RPA-T4) was obtained from BD Biosciences.
1 mg of anti-polymeric dye specific antibody (clones S37, S38) was labeled with Alexa647-NHS ester (5 μL, 10 mg/mL in DMSO) (Invitrogen), reacting for one hour. The dye conjugates were purified by gel filtration.

### B. Cell Staining and Flow Cytometry

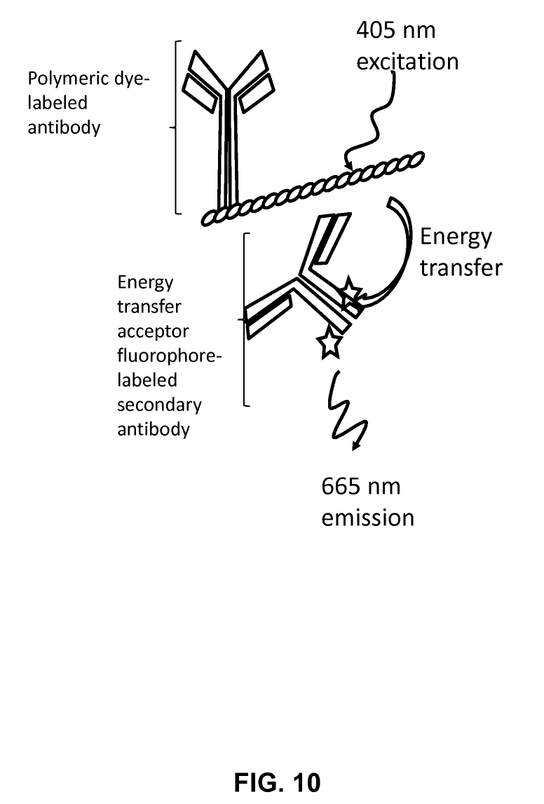

PBMCs were isolated using Ficoll-Paque density gradient media (1.6×10-6 cells/mL). The cells were stained with BV421-labelled anti-CD4 (4.2 μL reagent/1×10-6 cells) for 30 min. The cells were washed three times with staining buffer (1×PBS, 2% FBS, 0.09% sodium azide) and restained with S37-Alexa647 and S38-Alexa647 (0.2 μg/1×10-6 cells) for 30 min, and washed again before analysis on an LSRII flow cytometer (BD Biosciences). The data was processed using FACS Diva software (BD Biosciences). Alexa647 staining was observed, as predicted. However, Alexa647-emission was also observed upon excitation with 405 nm light. This observation was unexpected as 1) Alexa 647 has minimal excitation at this wavelength. As such, energy transfer was taking place between the polymeric dye and the Alexa647, e.g., as illustrated in FIG. 10. This phenomenon has utility as a signal amplification method or means of modulating the wavelengths of light emitted by a polymeric dye.

Notwithstanding the appended clauses, the disclosure set forth herein is also defined by the following clauses:

1. A proteinaceous specific binding member that specifically binds to a polymeric dye.
2. The specific binding member according to Clause 1, wherein the polymeric dye comprises a conjugated polymer comprising a plurality of first optically active units forming a conjugated system, having a first absorption wavelength at which the first optically active units absorbs light to form an excited state.
3. The specific binding member according to Clause 2, wherein the polymeric dye comprises the following structure:

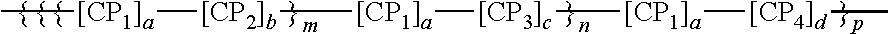

wherein CP₁, CP₂, CP₃ and CP₄ are independently a conjugated polymer segment or an oligomeric structure, wherein one or more of CP₁, CP₂, CP₃ and CP₄ are bandgap-lowering n-conjugated repeat units.
4. The specific binding member according to Clause 3, wherein the polymeric dye comprises one of the following structures:

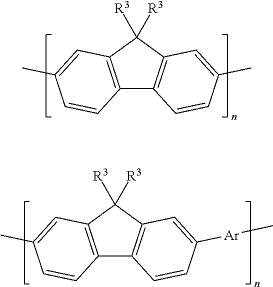

wherein each R³ is independently an optionally substituted alkyl or aryl group; Ar is an optionally substituted aryl or heteroaryl group; and n is 1 to 10000.
5. The specific binding member according to Clause 4, wherein the polymeric dye comprises the structure:

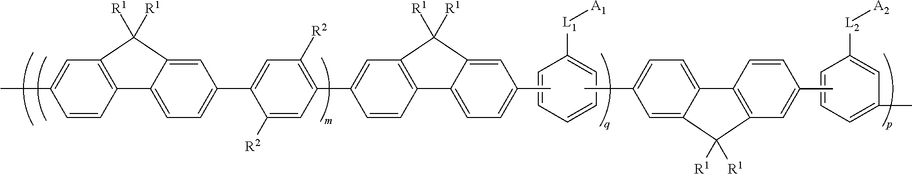

wherein:

each R¹ is independently a solubilizing group or a linker-dye;

L₁ and L₂ are optional linkers;

each R² is independently H or an aryl substituent; and

each A₁ and A₂ is independently H or a fluorophore.

6. The specific binding member according to any of Clauses 1 to 5, wherein the polymeric dye has an emission maximum selected from 421 nm, 510 nm, 570 nm, 602 nm, 650 nm, 711 nm and 786 nm.
7. The specific binding member according to any of Clauses 1 to 6, wherein the polymeric dye has an extinction coefficient of about 2×10⁶ or more and a quantum yield of about 0.5 or more.
8. The specific binding member according to Clause 6, wherein the specific binding member specifically binds the polymeric dye having an emission maximum of 421 nm.
9. The specific binding member according to Clause 6, wherein the specific binding member specifically binds to the polymeric dye having an emission maximum of 421 nm and the polymeric dye having an emission maximum of 510 nm.
10. The specific binding member according to Clause 6, wherein the specific binding member binds the polymeric dye having an emission maximum of 421 nm with a specificity of 5:1 or more over the polymeric dye having an emission maximum of 510 nm.
11. The specific binding member according to Clause 10, wherein the specific binding member has no cross-reactivity against the polymeric dye having an emission maximum of 510 nm.
12. The specific binding member according to any of the preceding clauses, wherein the polymeric dye is a polymeric tandem dye.
13. The specific binding member according to Clause 12, wherein the polymeric tandem dye comprises a polymeric dye linked to an acceptor fluorophore selected from the group consisting of Cy3, Cy3.5, Cy5, Cy5.5, Cy7, Alexa488, Alexa 647 and Alexa700.
14. The specific binding member according to any of the preceding clauses, wherein the specific binding member is selected from the group consisting of an antibody, a Fab fragment, a F(ab′)₂ fragment, a scFv, a diabody, or a triabody.
15. The specific binding member according to Clause 14, wherein the specific binding member is a murine antibody or binding fragment thereof.
16. The specific binding member according to Clause 14, wherein the specific binding member is recombinant antibody or binding fragment thereof.
17. A support bound proteinaceous specific binding member that specifically binds to a polymeric dye.
18. The specific binding member according to Clause 17, wherein the support is selected from the group consisting of a particle, a planar substrate, a fibrous mesh, a hydrogel, a porous matrix, a pin, a microarray surface and a chromatography support.
19. The specific binding member according to Clause 18, wherein the support comprises a magnetic particle.
20. The specific binding member according to any of Clauses 17 to 19, wherein the polymeric dye comprises a conjugated polymer comprising a plurality of first optically active units forming a conjugated system, having a first absorption wavelength at which the first optically active units absorbs light to form an excited state.
21. The specific binding member according to Clause 20, wherein the polymeric dye comprises the following structure:

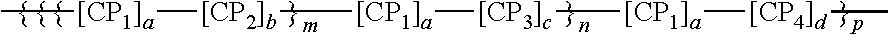

wherein CP₁, CP₂, CP₃ and CP₄ are independently a conjugated polymer segment or an oligomeric structure, wherein one or more of CP₁, CP₂, CP₃ and CP₄ are bandgap-lowering n-conjugated repeat units.
22. The specific binding member according to Clause 20, wherein the polymeric dye comprises one of the following structures:

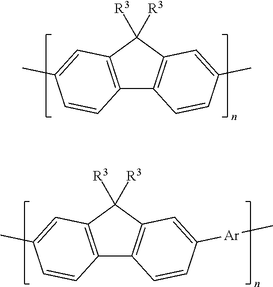

wherein each R³ is independently an optionally substituted alkyl or aryl group; Ar is an optionally substituted aryl or heteroaryl group; and n is 1 to 10000.
23. The specific binding member according to Clause 22, wherein the polymeric dye comprises the structure:

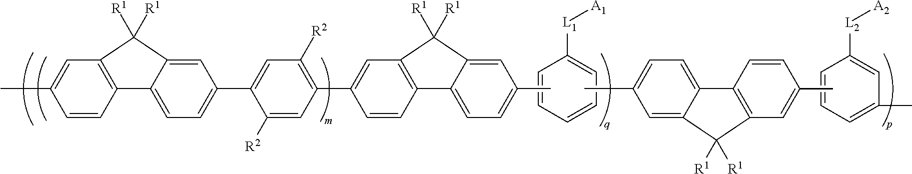

wherein:

each R¹ is independently a solubilizing group or a linker-dye;

L₁ and L₂ are optional linkers;

each R² is independently H or an aryl substituent; and

each A₁ and A₂ is independently H or a fluorophore.

24. The specific binding member according to any of Clauses 17 to 23, wherein the polymeric dye has an emission maximum selected from 421 nm, 510 nm, 570 nm, 602 nm, 650 nm, 711 nm and 786 nm.
25. The specific binding member according to any of Clauses 17 to 24, wherein the polymeric dye has an extinction coefficient of 2×10⁶ or more and a quantum yield of 0.5 or more.
26. The specific binding member according to Clause 24, wherein the specific binding member specifically binds the polymeric dye having an emission maximum of 421 nm.
27. The specific binding member according to Clause 24, wherein the specific binding member specifically binds to the polymeric dye having an emission maximum of 421 nm and the polymeric dye having an emission maximum of 510 nm.
28. The specific binding member according to Clause 17, wherein the specific binding member binds the polymeric dye having an emission maximum of 421 nm with a specificity of 5:1 or more over the polymeric dye having an emission maximum of 510 nm.
29. The specific binding member according to Clause 28, wherein the specific binding member has no cross-reactivity against the polymeric dye having an emission maximum of 510 nm.
30. The specific binding member according to any of Clauses 17 to 29, wherein the polymeric dye is a polymeric tandem dye.
31. The specific binding member according to Clause 30, wherein the polymeric tandem dye comprises a polymeric dye linked to an acceptor fluorophore selected from the group consisting of Cy3, Cy3.5, Cy5, Cy5.5, Cy7, Alexa488, Alexa 647 and Alexa700.
32. The specific binding member according to any of Clauses 17 to 31, wherein the specific binding member is selected from the group consisting of an antibody, a Fab fragment, a F(ab′)₂ fragment, a scFv, a diabody, or a triabody.
33. The specific binding member according to Clause 32, wherein the specific binding member is a murine antibody or binding fragment thereof.
34. The specific binding member according to Clause 32, wherein the specific binding member is recombinant antibody or binding fragment thereof.
35. A method of separating a polymeric dye-labeled target from a sample, the method comprising:

(a) contacting a sample comprising a polymeric dye-labeled target with a proteinaceous specific binding member that specifically binds to the polymeric dye of the polymeric dye-labeled target to form a complex; and

(b) separating the complex from the sample.

36. The method according to Clause 35, further comprising detecting the polymeric dye-labeled target.
37. The method according to Clause 35, further comprising analyzing the polymeric dye-labeled target.
38. The method according to Clause 36, wherein the dye labeled target is a nucleic acid, a protein, a peptide, a cell or a small molecule.
39. The method according to any of Clauses 35 to 38, wherein the polymeric dye-labeled target comprises a target specifically bound to a polymeric dye-labeled affinity agent.
40. The method according to any of Clauses 35 to 38, wherein the polymeric dye-labeled target comprises a target covalently bound to a polymeric dye.
41. The method according to any of Clauses 35 to 40, wherein the proteinaceous specific binding member is support bound.
42. The method according to Clause 41, wherein the support is selected from the group consisting of a particle, a solid substrate, a fibrous mesh, a hydrogel, a porous matrix, a pin, a microarray surface and a chromatography support.
43. The method according to Clause 42, wherein the support comprises a magnetic particle.
44. The method according to Clause 43, wherein the separating comprises applying an external magnetic field to immobilize the magnetic particle.
45. The method according to any of Clauses 41 to 44, wherein the separating further comprises washing the support to remove unbound material of the sample.
46. A method of separating a polymeric dye-labeled cell from a sample, the method comprising:

(c) contacting a sample comprising a polymeric dye-labeled cell with a support bound proteinaceous specific binding member that specifically binds to the polymeric dye of the polymeric dye-labeled cell;

(d) separating the support from the sample; and

(e) separating the polymeric dye-labeled cell from the support using a biocompatible aqueous eluent.

47. The method according to Clause 46, wherein the polymeric dye-labeled cell comprises a target cell specifically bound to a polymeric dye-labeled affinity agent.
48. The method according to Clause 47, wherein the method further comprises contacting the target cell with the polymeric dye-labeled affinity agent to produce the polymeric dye-labeled cell.
49. The method according to any of Clauses 46 to 48, wherein the support is selected from the group consisting of a particle, a solid substrate, a fibrous mesh, a hydrogel, a porous matrix, a pin, a microarray surface and a chromatography support.
50. The method according to Clause 49, wherein the support comprises a magnetic particle.
51. The method according to Clause 50, wherein the separating comprises applying an external magnetic field to immobilize the magnetic particle.
52. The method according to any of Clauses 46 to 51, wherein the separating further comprises washing the support to remove unbound material of the sample.
53. The method according to any of Clauses 46 to 52, wherein the method further comprises detecting the polymeric dye-labeled cell.
54. The method according to any of Clauses 46 to 53, wherein the method further comprises flow cytometrically analyzing the polymeric dye-labeled cell.
55. The method according to Clause 48, wherein the target cell comprises a cell surface marker selected from the group consisting of a cell receptor and a cell surface antigen.
56. The method according to any of Clauses 46 to 55, wherein the biocompatible aqueous eluent is non-cytotoxic and non-denaturing to the polymeric dye-labeled cell.
57. The method according to Clause 56, wherein the biocompatible aqueous eluent comprises a polyethylene glycol.
58. A method of determining whether a cell is present in a sample, the method comprising:

(a) contacting a sample suspected of comprising a polymeric dye-labeled cell with a support bound proteinaceous specific binding member that specifically binds to the polymeric dye of the polymeric dye-labeled cell;

(b) separating the support from the sample;

(c) subjecting the support to elution conditions comprising a biocompatible aqueous elution buffer to produce an eluent; and

(d) evaluating whether the isolated polymeric dye-labeled cell is present in the eluent to determine whether a cell is present in a sample.

59. The method according to Clause 58, wherein the evaluating comprises flow cytometrically analyzing the eluent.
60. The method according to Clauses 58 or 59, further comprising, prior to the contacting, combining a sample suspected of comprising a target cell with a polymeric dye-specific binding member conjugate.
61. The method according to any of Clauses 58 to 60, wherein the support is a magnetic particle and the separating comprises applying an external magnetic field.
62. A method of analyzing a cell, the method comprising:

(a) contacting a sample comprising a polymeric dye-labeled cell with a support bound proteinaceous specific binding member that specifically binds to the polymeric dye of the polymeric dye-labeled cell;

(b) separating the support from the sample;

(c) dissociating the polymeric dye-labeled cell from the support using a biocompatible aqueous elution buffer to produce an eluent comprising the dissociated cell; and

(d) flow cytometrically analyzing the dissociated cell.

63. The method according to Clause 62, further comprising, prior to the contacting, combining a sample comprising a target cell with a polymeric dye-specific binding member conjugate to produce the polymeric dye-labeled cell.
64. The method according to Clauses 62 or 63, wherein the support comprises a magnetic particle and the separating comprises applying an external magnetic field.
65. A kit comprising:

a proteinaceous specific binding member that specifically binds to a polymeric dye; and

one or more components selected from the group consisting of a polymeric dye, a polymeric tandem dye, a polymeric dye-specific binding member conjugate, a cell, a support, an biocompatible aqueous elution buffer, and instructions for use.

66. The kit according to Clause 65, wherein the proteinaceous specific binding member is support bound.
67. The kit according to Clause 66, wherein the support is selected from the group consisting of a particle, a solid substrate, a fibrous mesh, a hydrogel, a porous matrix, a pin, a microarray surface and a chromatography support.
68. The kit according to Clause 67, wherein the support comprises a magnetic particle.
69. A composition comprising:

a polymeric dye; and

a proteinaceous specific binding member that specifically binds to the polymeric dye.

70. A composition comprising:

a polymeric dye-labeled cell; and

a proteinaceous specific binding member that specifically binds to the polymeric dye of the polymeric dye-labeled cell.

71. The composition according to Clause 70, wherein the proteinaceous specific binding member is support bound.
72. The composition according to Clause 71, wherein the support is selected from the group consisting of a particle, a solid substrate, a fibrous mesh, a hydrogel, a porous matrix, a pin, a microarray surface and a chromatography support.
73. The composition according to Clause 72, wherein the support comprises a magnetic particle.
74. The composition according to any of Clauses 71 to 73, wherein the polymeric dye comprises a conjugated polymer comprising a plurality of first optically active units forming a conjugated system, having a first absorption wavelength at which the first optically active units absorbs light to form an excited state.
75. The composition according to Clause 74, wherein the polymeric dye has an emission maximum selected from 421 nm, 510 nm, 570 nm, 602 nm, 650 nm, 711 nm and 786 nm.
76. The composition according to Clause 75, wherein the polymeric dye has an emission maximum of 421 nm or 510 nm.
77. The composition according to any of Clauses 70 to 74, wherein the polymeric dye is a polymeric tandem dye.
78. A composition comprising:

a polymeric dye-labeled antibody that specifically binds a target cell; and

a proteinaceous specific binding member that specifically binds to the polymeric dye.

79. The composition according to Clause 78, wherein the proteinaceous specific binding member is support bound.
80. The composition according to Clause 79, wherein the support is selected from the group consisting of a particle, a solid substrate, a fibrous mesh, a hydrogel, a porous matrix, a pin, a microarray surface and a chromatography support.
81. The composition according to Clause 80, wherein the support comprises a magnetic particle.
82. The composition according to any of Clauses 78 to 81, wherein the polymeric dye-labeled antibody comprises a conjugated polymer comprising a plurality of first optically active units forming a conjugated system, having a first absorption wavelength at which the first optically active units absorbs light to form an excited state.
83. The composition according to any of Clauses 78 to 82, wherein the target cell comprises a cell surface marker selected from a cell receptor and a cell surface antigen.
84. The composition according to any of Clauses 78 to 83, wherein the antibody specifically binds a polymeric dye having an emission maximum of 421 nm or a polymeric dye having an emission maximum of 510 nm.
85. The composition according to Clause 84, wherein the polymeric dye is a polymeric tandem dye.
86. A composition comprising:

a cell-containing biological sample;

a polymeric dye-specific binding member conjugate that specifically binds a target cell; and

a proteinaceous specific binding member that specifically binds to the polymeric dye.

87. The composition according to Clause 86, wherein the proteinaceous specific binding member is support bound.
88. The composition according to Clause 87, wherein the support is selected from the group consisting of a particle, a solid substrate, a fibrous mesh, a hydrogel, a porous matrix, a pin, a microarray surface and a chromatography support.
89. The composition according to Clause 88, wherein the support comprises a magnetic particle.
90. The composition according to any of Clauses 86 to 89, wherein the polymeric dye comprises a conjugated polymer comprising a plurality of first optically active units forming a conjugated system, having a first absorption wavelength at which the first optically active units absorbs light to form an excited state.
91. The composition according to Clause 90, wherein the polymeric dye has an emission maximum selected from 421 nm, 510 nm, 570 nm, 602 nm, 650 nm, 711 nm and 786 nm.
92. The composition according to Clause 91, wherein the polymeric dye has an emission maximum of 421 nm or 510 nm.
93. The composition according to Clause 91, wherein the polymeric dye is a polymeric tandem dye.
94. A flow cytometric system, comprising:

a flow cytometer comprising a flow path;

a composition in the flow path, wherein the composition comprises:
a cell-containing biological sample;
a polymeric dye-specific binding member conjugate that specifically binds a target cell; and
a support bound proteinaceous specific binding member that specifically binds to the polymeric dye
   95. The flow cytometric system according to Clause 94, wherein the sample comprises a polymeric dye-labeled cell comprising the polymeric dye-specific binding member conjugate specifically bound to a target cell.
   96. The flow cytometric system according to Clause 94, wherein the support comprises a magnetic particle.
   97. The flow cytometric system according to Clause 94, wherein the polymeric dye comprises a conjugated polymer comprising a plurality of first optically active units forming a conjugated system, having a first absorption wavelength at which the first optically active units absorbs light to form an excited state.
   98. The flow cytometric system according to Clause 97, wherein the polymeric dye has an emission maximum selected from 421 nm, 510 nm, 570 nm, 602 nm, 650 nm, 711 nm and 786 nm.
   99. The flow cytometric system according to Clause 98, wherein the polymeric dye has an emission maximum of 421 nm or 510 nm.
   100. The flow cytometric system according to Clause 97, wherein the polymeric dye is a polymeric tandem dye.
   101. A method of evaluating whether an analyte is present in a sample, the method comprising:
   (a) contacting a sample with a signal producing system comprising proteinaceous specific binding member that specifically binds to a polymeric dye;
   (b) assaying the sample for a signal from the signal producing system to obtain a result; and
   (c) evaluating whether the analyte is present in the sample based on the result.
   102. The method according to Clause 101, wherein the proteinaceous specific binding member is a proteinaceous specific binding member according to any of Clauses 1 to 16.
   103. The method according to Clauses 101 or 102, wherein the proteinaceous specific binding member is labeled with an acceptor moiety.
   104. The method according to Clauses 101 or 102, wherein proteinaceous specific binding member is labeled with an indirectly detectable label.
   105. The method according to any of Clauses 101 to 104, wherein the analyte is a cell.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the following.

What is claimed is:

1. A proteinaceous specific binding member that specifically binds to a polymeric dye.

2. The specific binding member according to claim 1, wherein the polymeric dye comprises a conjugated polymer comprising a plurality of first optically active units forming a conjugated system, having a first absorption wavelength at which the first optically active units absorbs light to form an excited state.

3. The specific binding member according to claim 2, wherein the polymeric dye comprises the following structure: 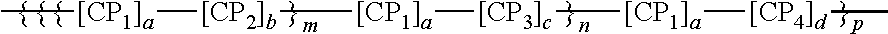
wherein CP₁, CP₂, CP₃ and CP₄ are independently a conjugated polymer segment or an oligomeric structure, wherein one or more of CP₁, CP₂, CP₃ and CP₄ are bandgap-lowering n-conjugated repeat units.

4. The specific binding member according to any of claims 1 to 3, wherein the polymeric dye has an extinction coefficient of about 2×10⁶ or more and a quantum yield of about 0.5 or more.

5. The specific binding member according to any of the preceding claims, wherein the polymeric dye is a polymeric tandem dye.

6. The specific binding member according to any of the preceding claims, wherein the specific binding member is selected from the group consisting of an antibody, a Fab fragment, a F(ab′)₂ fragment, a scFv, a diabody, or a triabody.

7. The specific binding member according to any of the preceding claims, wherein the specific binding member is support bound.

8. The specific binding member according to claim 7, wherein the support is selected from the group consisting of a particle, a planar substrate, a fibrous mesh, a hydrogel, a porous matrix, a pin, a microarray surface and a chromatography support.

9. The specific binding member according to claim 8, wherein the support comprises a magnetic particle.

10. A method comprising contacting a sample with a specific binding member according to any of the preceding claims.

11. The method according to claim 10, wherein the method is a method of separating a polymeric dye-labeled target from a sample.

12. The method according to claim 10, wherein the method is a method of evaluating the sample for the presence of an analyte.

13. The method according to any of claims 11 and 12, wherein the target or analyte are cells.

14. A kit comprising:
a proteinaceous specific binding member that specifically binds to a polymeric dye; and
one or more components selected from the group consisting of a polymeric dye, a polymeric tandem dye, a polymeric dye-specific binding member conjugate, a cell, a support, an biocompatible aqueous elution buffer, and instructions for use.

15. A flow cytometric system, comprising:
a flow cytometer comprising a flow path;
a composition in the flow path, wherein the composition comprises:
a cell-containing biological sample; and
a polymeric dye-specific binding member conjugate that specifically binds a target cell.
